(19) United States
(12) Patent Application Publication
(10) Pub. No.: US 2026/0335450 A1
(43) Pub. Date: Oct. 29, 2026

(54) TARGETED PROTEIN DEGRADATION THROUGH SITE-SPECIFIC RECRUITMENT OF UBIQUITIN LIGASE

(71) Applicant: University of Pittsburgh - Of the Commonwealth System of Higher Education, Pittsburgh, PA (US)

(72) Inventors: Alexander Deiters, Pittsburgh, PA (US); Olivia Hanson Shade, Pittsburgh, PA (US)

(73) Assignee: University of Pittsburgh - Of the Commonwealth System of Higher Education, Pittsburgh, PA (US)

(21) Appl. No.: 19/486,314

(22) PCT Filed: May 21, 2024
(86) PCT No.: PCT/US2024/030435

§ 371 Date: Nov. 20, 2025

Related U.S. Application Data
(60) Provisional application No. 63/470,110, filed on May 31, 2023.

(51) Int. Cl. A61K 47/55

(52) U.S. Cl. CPC A61K 47/55

(57) ABSTRACT

A compound, or a stereoisomer, isotopomer, tautomer, or salt thereof having a structure of:

A-L-B

wherein A is a ligand that can covalently bioconjugate to an unnatural amino acid bearing a tetrazine or other reactive handle, incorporated into a protein; L is a linker covalently attaching A and B; and B is a ligand that can bind to a E3 ligase.

### CROSS REFERENCE TO RELATED APPLICATION

This application claims the benefit of U.S. Provisional Application No. 63/470,110, filed May 31, 2023, which is herein incorporated by reference in its entirety.

### ACKNOWLEDGMENT OF GOVERNMENT SUPPORT

This invention was made with government support under Grant Number 1904972 awarded by the National Science Foundation. The government has certain rights in the invention.

### BACKGROUND

Targeted protein degradation, or the use of small molecules to redirect native degradation machinery, allows for rapid and reversible protein knockdown. The most commonly applied tools for targeted protein degradation are proteolysis targeting chimeras (PROTACs), which are heterobifunctional small molecule degraders containing a ligand (green star) specific to a protein of interest (POI, orange) tethered via a chemical linker to an E3 ligase ligand (purple triangle, FIG. 2A). This design induces recruitment of the native E3 ligase to the POI, thus promoting the ubiquitination and subsequent proteasomal degradation of the target via the Ubiquitin Proteasome System (UPS). The ligand for the POI is typically derived from a known, high affinity, small molecule ligand. By exploiting the UPS, PROTACs provide several advantages over classical small molecule inhibition, namely the lessened restraints of POI binding location, as protein function does not need to be altered, and the prolonged response of degradation as compared to inhibition alone, thus expanding the realm of “druggable” proteins.

### SUMMARY

Disclosed herein is the site-specific recruitment of native ubiquitin ligases to a protein of interest via bioorthogonal E3 ligase ligands. This novel methodology will provide insight into overall protein degradability and streamline the process for ligand and PROTAC development to achieve optimal protein degradation. Furthermore, the approach represents conditional OFF switching of protein function through small molecule-triggered degradation. In certain embodiments the protein of interest is only modified with a minimal surface modification (<200 Da) and does not require any domain fusions.

In one aspect, there is disclosed herein a compound, or a stereoisomer, isotopomer, tautomer, or salt thereof having a structure of:

A-L-B

wherein A is a ligand that can covalently bioconjugate to a tetrazine-modified unnatural amino acid expressed on a protein or to a dienophile-modified unnatural amino acid expressed on a protein; L is a linker covalently attaching A and B; and B is a ligand that can bind to a E3 ligase.

In another aspect, there is disclosed herein a method comprising:
covalently bioconjugating the A-L-B compound to a protein of interest wherein the protein of interest includes a site-specific installed conjugation handle for the compound.

The foregoing will become more apparent from the following detailed description, which proceeds with reference to the accompanying figures.

### BRIEF DESCRIPTION OF THE DRAWINGS

FIGS. 1A-1B. Experimental workflow for the quantification of bioconjugation to UAA-bearing proteins expressed in cells. Following expression of the POI bearing a UAA at any surface position (e.g., A-E), the FIG. 1A) sTCO-chloroalkane ligand or FIG. 1B) sTCO-E3 ligase ligand, is added to the media and undergoes a selective bioconjugation with the UAA. Excess ligand is washed away and cells are FIG. 1A) lysed or FIG. 1B) incubated with sTCO-chloroalkane then lysed. The lysate is incubated with HaloTag protein and the bioconjugation is quantified via western blot.

FIGS. 2A-2D. FIG. 2A) Mechanism of action of small molecule PROTACs. FIG. 2B) Site-specific conjugation of an E3 ligase ligand for targeted protein degradation. FIG. 2C) TetF amino acid site-specifically installed at defined positions of the POI surface in human cells with an expanded genetic code. FIG. 2D) Structure of E3 ligase ligand, cereblon (CRBN) modified with an sTCO handle for tetrazine ligation via an inverse-electron demand Diels-Alder reaction, where n=2 or 9.

FIGS. 3A-3E. Optimization of bioorthogonal E3 ligase mediated degradation in mammalian cells. Effect of increasing concentrations of sTCO-PEG₂-CRBN on mCherry-EGFP-Y151TetF-HA after overnight (FIG. 3A), one hour (FIG. 3B), or 15 min (FIG. 3C) exposure to the compound, each incubated overnight. (FIG. 3D) Time course of sTCO-PEG₂-CRBN (10 μM) mediated degradation of mCherry-EGFP-Y151TetF, as determined by two biological replicates analyzed by western blot. (FIG. 3E) Comparison of the effect of PEG-length on degradation ability via the dose response of mCherry-EGFP-Y151TetF treated with increasing concentrations of sTCO-PEG₉-CRBN. A-C, and E each completed in biological triplicate, with statistical significance determined by one-way ANOVA relative to the negative control, where *=p<0.1, **=p<0.01, ***=p<0.001, and ****=p<0.0001.

FIGS. 4A-4D. FIG. 4A) Crystal structure of EGFP with mutated residues highlighted in orange and lysine residues highlighted in blue (PDB 2Y0G). FIG. 4B) Representative western blots of mCherry-EGFP-TetF mutants following treatment with sTCO-PEG₂-CRBN or sTCO-PEG₉-CRBN. Full blots are available in FIG. 9. Quantification of FIG. 4C) sTCO-PEG₂-CRBN. and FIG. 4D) sTCO-PEG₉-CRBN conjugation versus degradation observed for each EGFP-TetF mutant, with percentage of degradation per bioconjugated protein listed below each mutant. Percent conjugation was determined by pulse-chase analysis. Error bars were calculated as standard deviation from the mean of three biological replicates with statistical significance determined via one-way ANOVA, where **=p<0.01 and ****=p<0.0001.

FIGS. 5A-5D. FIG. 5A) Crystal structure of DUSP6 with mutated residues highlighted in orange and lysine residues highlighted in blue (PDB 1MKP), docked with the allosteric ligand BCI (green structure). FIG. 5B) Representative western blots of DUSP6-TetF mutants following treatment with sTCO-PEG₂-CRBN or sTCO-PEG₉-CRBN. Full blots are available in FIG. 12. Quantification of FIG. 5C) sTCO-PEG₂-CRBN and FIG. 5D) sTCO-PEG₉-CRBN conjugation versus degradation observed for each DUSP6-TetF mutant, with percentage of degradation per bioconjugated protein listed below each mutant. Percent conjugation was determined by pulse-chase analysis. Error bars were calculated as standard deviation from the mean of three biological replicates with statistical significance determined via one-way ANOVA, where **=p<0.01, ***=p<0.001, and ****=p<0.0001.

FIGS. 6A-6D. FIG. 6A) Crystal structure of ERRα with mutated residues highlighted in orange, lysines highlighted in blue, co-crystalized with the known ERRα ligand in dark green (PDB 3K6P). FIG. 6B) Representative western blots of ERRα-TetF mutants following treatment with sTCO-PEG₂-CRBN or sTCO-PEG₉-CRBN. Full blots are available in FIG. 15. Quantification of FIG. 6C) sTCO-PEG₂-CRBN and FIG. 6D) sTCO-PEG₉-CRBN conjugation versus degradation observed for each ERRα-TetF mutant, with percentage of degradation per bioconjugated protein listed below each mutant. Percent conjugation was determined by pulse-chase analysis. Error bars were calculated as standard deviation from the mean of three biological replicates with statistical significance determined via one-way ANOVA, where *=p<0.1 and **=p<0.01.

FIG. 7. Quantification of Bioconjugation to mCherry-EGFP-TetF mutants. The extent of bioconjugation to new mCherry-EGFP mutants G191TetF and A206TetF were determined by incubating cells expressing the respective TetF-bearing protein with sTCO-CA (10 μM) for 2 hours. Following cell lysis, the lysate was incubated with recombinant HaloTag and the resultant conjugate formation was observed and quantified via HA and GAPDH western blots.

FIGS. 8A-8F. Pulse-Chase Quantification for mCherry-EGFP-TetF mutants. The bioconjugation efficiency of sTCO-PEG₂-CRBN and sTCO-PEG₉-CRBN as determined via triplicate pulse-chase experiment for FIG. 8A) mCherry-EGFP-Y151TetF-HA, FIG. 8B) mCherry-EGFP-I129TetF-HA, FIG. 8C) mCherry-EGFP-G134TetF-HA, FIG. 8D) mCherry-EGFP-G191TetF-HA, and FIG. 8E) mCherry-EGFP-A206TetF-HA. FIG. 8F) Percentage of bioconjugation calculated for each compound.

FIGS. 9A-9B. Full HA and GAPDH blots for each quantification shown in FIG. 4B-C. Statistical analysis of three biological replicates. Error bars were calculated from three biological replicates, with statistical significance determined through one-way ANOVA, where **=p<0.01, ***=p<0.001, and ****=p<0.0001.

FIG. 10. Quantification of sTCO-CA Bioconjugation to DUSP6-TetF mutants. The extent of bioconjugation to DUSP6-HA mutants L246TetF, S265TetF, S277TetF, and G338TetF were determined by incubating cells expressing the respective TetF-bearing protein with sTCO-CA (10 μM) for 2 hours. Following cell lysis, the lysate was incubated with recombinant HaloTag and the resultant conjugate formation was observed and quantified via HA and GAPDH western blots.

FIGS. 11A-11E. Pulse-Chase Quantification for DUSP6-TetF mutants. The bioconjugation efficiency of sTCO-PEG₂-CRBN and sTCO-PEG₉-CRBN as determined via triplicate pulse-chase experiments for FIG. 11A) DUSP6-L246TetF-HA, FIG. 11B) DUSP6-S265TetF-HA, FIG. 11C) DUSP6-S277TetF-HA, and FIG. 11D) DUSP6-G338TetF-HA. FIG. 11E) Percentage of bioconjugation calculated for each compound.

FIGS. 12A-12B. Full HA and GAPDH blots for each quantification shown in FIG. 5B. Error bars were calculated from three biological replicates, with statistical significance determined through one-way ANOVA or paired t-test, where ***=p<0.001, and ****=p<0.0001.

FIG. 13. A Quantification of sTCO-CA Bioconjugation to ERRα-TetF mutants. The extent of bioconjugation to ERRα-HA mutant H437TetF was determined by incubating cells expressing the TetF-bearing protein with sTCO-CA (10 μM) for 2 hours. Following cell lysis, the lysate was incubated with recombinant HaloTag and the resultant conjugate formation was observed and quantified via HA and GAPDH western blots.

FIGS. 14A-14E. Pulse-Chase Quantification of ERRα-TetF Mutants B) The bioconjugation efficiency of sTCO-PEG₂-CRBN and sTCO-PEG₉-CRBN as determined via triplicate pulse-chase experiments for FIG. 14A) ERRα-V299TetF-HA, FIG. 14B) ERRα-F382TetF-HA, FIG. 14C) ERRα-H437TetF-HA, and FIG. 14D) ERRα-F495TetF-HA. FIG. 14E) Percentage of bioconjugation calculated for each compound.

FIGS. 15A-15B. Full HA and GAPDH blots for each quantification shown in FIG. 6B. Error bars were calculated from three biological replicates, with statistical significance determined through one-way ANOVA or paired t-test, where *=p<0.1, **=p<0.01, and ***=p<0.001.

FIGS. 16A-16B. Degradation mechanism of sTCO-PEG₂-CRBN. FIG. 16A) The degradation of mCherry-EGFP-Y151TetF was reduced when cells were pre-treated with proteasome inhibitor MG132 or epoxomicin, validating that sTCO-PEG₂-CRBN induced degradation is mediated by the proteasome. FIG. 16B) Pretreatment of cells expressing mCherry-EGFP-Y151TetF with 100 UM of thalidomide-OH (Thal-OH) followed by treatment with sTCO-PEG₂-CRBN reduced degradation, validating cereblon-mediated degradation.

FIG. 17. Specificity of sTCO-PEG₂-CRBN for TetF. No background reactivity was observed upon treatment of wild-type mCherry-EGFP-HA with sTCO-PEG₂-CRBN.

### DETAILED DESCRIPTION

While classical PROTACs have become invaluable tools for studying previously “undruggable” targets, their successful design requires prior knowledge of a small molecule ligand specific to the POI. PROTACs are further limited by the generality of the binding location of the known ligand, as the ligand pocket may not be proximal to surface lysine residues necessary for ubiquitination and subsequent degradation via the UPS. As the scope of PROTACs continues to evolve, a generalizable method for bottom-up PROTAC design that accounts for lysine proximity would be invaluable toward the development of novel degraders for any POI. Thus, disclosed herein is a tool that allows for the precise, site-specific recruitment of an E3 ligase ligand to any protein of interest—even those without an established small molecule ligand. Utilizing standard unnatural amino acid (UAA) mutagenesis to infer bioorthogonal reactivity to a protein surface, this novel approach can elucidate optimal locations for PROTAC binding based on the surface properties for any protein. This tool allows for (1) targeted degradation of POIs without any known small molecule ligands, (2) targeted degradation of highly promiscuous POIs, (3) elucidation of optimal protein surface microenvironments for improving classical PROTAC designs, and (4) a potential to guarantee E3 ligase recruitment to a POI by reversing our methodology.

Numerous approaches exist to modify native proteins in cellulo, however, the only way to ensure precise and freely customizable site-selectivity across a protein surface is via unnatural amino UAA mutagenesis. Genetic code expansion utilizes an aminoacyl-tRNA synthetase and tRNA_{CUA} pair evolved from orthogonal systems. The pyrrolysyl-tRNA synthetase derived from M. barkeri or M. mazei is favored for its orthogonality in both eukaryotic and bacterial cells. The synthetase/tRNA pair then encodes the desired UAA in response to an amber stop codon (UAG), and has been engineered to encode more than 100 unique amino acids. A large class of UAAs is dedicated to conferring bioorthogonal reactivity to a protein surface. Bioorthogonal UAAs have been synthesized and applied for a variety of bioorthogonal chemistries: azides and phosphines for Staudinger ligations, azides and alkynes for the copper(I)-catalyzed azide-alkyne cycloaddition (CuAAC), azides and strained alkynes for strain-promoted azide-alkyne cycloadditions (SPAAC), or tetrazines and dienophiles for inverse electron-demand Diels-Alder cycloadditions (IEDDA). The fast kinetics, small reactive handles, minimal toxicity, and superior efficiency of IEDDA reactions make this method the favored approach for in cellulo bioconjugations.

Protein bioconjugation—specific covalent bond formation between a protein of interest (POI) and a label—has a major impact on protein imaging, protein function studies, and drug discovery. Ideal protein bioconjugation should be fast, quantitative, one step with no off-target binding, and have minimal impact on protein function, no matter the complexity of the biological environment. The impressive selectivity and kinetics of bioorthogonal reactions, specifically the catalyst-free inverse-electron demand Diels-Alder (IEDDA) cycloaddition of tetrazines and strained alkene dienophiles, make IEDDA-reactive unnatural amino acids an exceptional tool for protein bioconjugation via genetic code expansion. Analyses of these bioconjugation reactions in live cells has been mostly limited to fluorophore labeling, and thus is rarely quantitative. Self-labeling enzymes, like HaloTag, have been used for protein isolation and purification, studies of protein-protein interactions, and in vivo and in vitro imaging. The excellent permeability and low reactivity of the chloroalkane substrate of HaloTag yields bioconjugation detection independent of ligand permeability. Utilizing the HaloTag technology in combination with genetic code expansion, a generalized method for quantification of intracellular bioconjugation to any POI at defined sites has been developed as disclosed herein.

Bioconjugation efficiency can assessed through site-specific installation of a reactive handle on a POI, followed by ligand-mediated protein labeling via ternary complex formation after cell lysis and exposure to HaloTag. Described herein are concentrations, reaction times, and reaction partners for bioconjugation and pulldown for POIs expressing different IEDDA reactive surface residues. Also disclosed herein is the application of this method for labeling protein targets, such as kinases and nuclear receptors. Quantitative assessment of protein bioconjugation reactions in live cells has been demonstrated.

For example, the direct comparison of degradation ability of proteins expressing the requisite unnatural amino acid at different regions of protein surface is detailed below. We demonstrate the requirement for proximal lysine residues and investigate flexibility in linker length for the design of optimal degraders to improve PROTAC design pipelines. Two proteins without known small molecule ligands, fluorescent reporter EGFP and MAPK phosphatase 3 (MKP3), were expressed bearing tetrazine-phenylalanine at varying surface locations and differentially degraded via bioconjugation with a strained trans-cyclooctene connected via PEG-linker to the cereblon E3 ligase ligand. Further, the degradation of a known PROTAC target ERRα was improved through the bioorthogonal recruitment of the E3 ligase to regions apart from the known ligand binding site.

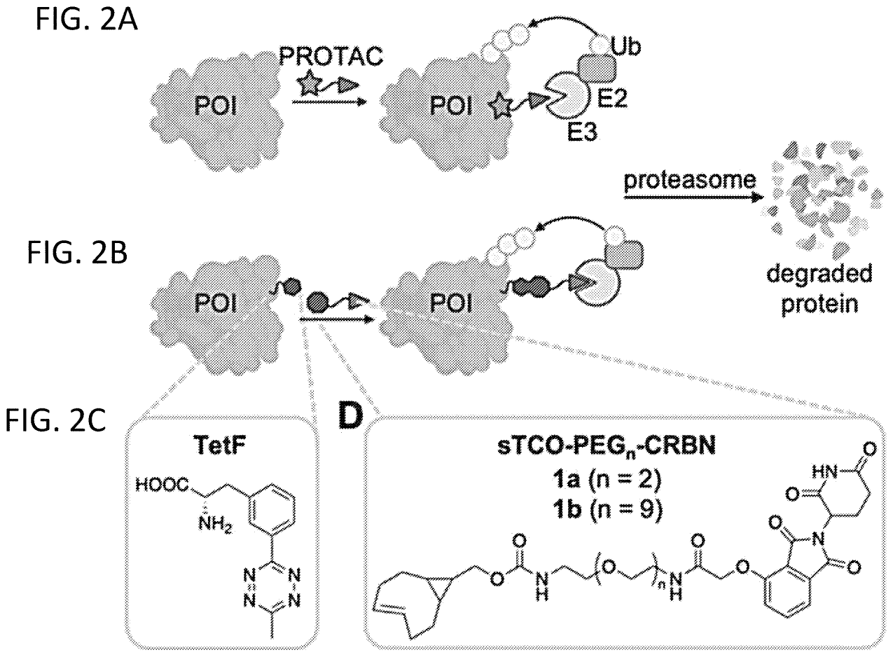

To combine the advantages of site specificity afforded from genetic code expansion with the potency of PROTACs for targeted degradation, a system has been designed that utilizes a bioorthogonal E3 ligase ligand for the site-specific, completely selective, degradation of proteins (see, for example, FIG. 2B). The compounds and methods disclosed herein provide at least the following advantages: the E3 ligase ligand can be freely moved across the surface of the protein (i.e., it can be attached at any surface site); the attachment to the protein of interest is covalent, so there is no concern about binding affinity; does not rely on a known small molecule ligand; applicable to any protein and in any cell type; applicable to any organism with an expanded genetic code, such as cells, C. elegans, drosophila, zebrafish, and xenopus. The compounds and methods disclosed herein provide a system for testing of the PROTACability of a protein of interest even before a small molecule ligand is discovered; if the protein can be degraded, the system can identify ideal sites for ligand discovery to achieve the most efficient degradation.

One aspect of the system disclosed herein is a compound of formula I, or a stereoisomer, isotopomer, tautomer, or salt thereof:

A-L-B

wherein A is a ligand that can covalently bioconjugate to a tetrazine-modified unnatural amino acid or to a dienophile-modified unnatural amino acid; L is a linker covalently attaching A and B; and B is a ligand that can bind to a E3 ligase.

Another aspect of the system disclosed herein is a compound, or a stereoisomer, isotopomer, tautomer, or salt thereof having a structure of:

A-L-B

wherein A is a ligand that can covalently bioconjugate to a tetrazine-modified unnatural amino acid, or to a dienophile-modified unnatural amino acid, or to an azide-modified unnatural amino acid; L is a linker covalently attaching A and B; and B is a ligand that can bind to an E3 ligase.

Another aspect of the system disclosed herein is a compound, or a stereoisomer, isotopomer, tautomer, or salt thereof having a structure of:

A-L-B

wherein A is a ligand that can covalently bioconjugate to a tetrazine-modified unnatural amino acid expressed on a protein, or to a dienophile-modified unnatural amino acid expressed on a protein, or to an azide-modified unnatural amino acid expressed on a protein; L is a linker covalently attaching A and B; and B is a ligand that can bind to an E3 ligase.

Another aspect of the system disclosed herein is a compound, or a stereoisomer, isotopomer, tautomer, or salt thereof having a structure of:

A-L-B

wherein A is a ligand that can covalently bioconjugate to a tetrazine-modified unnatural amino acid present on a protein, or to a dienophile-modified unnatural amino acid present on a protein, or to an azide-modified unnatural amino acid present on a protein; L is a linker covalently attaching A and B; and B is a ligand that can bind to an E3 ligase.

In certain embodiments B is a ligand for CRBN. In certain embodiments B is a ligand for VHL.

Ligand B may include a thalidomide analog for recruiting CRBN. Illustrative analogs include:

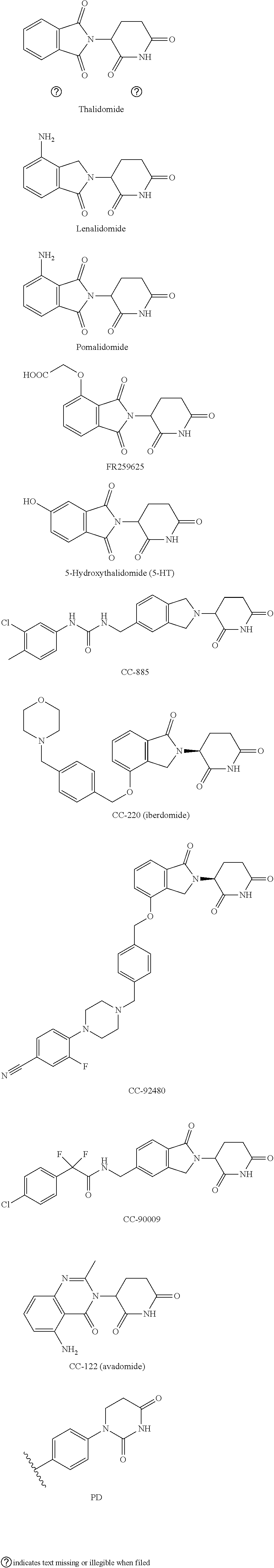

Additional E3 ligase ligands for CRBN include:

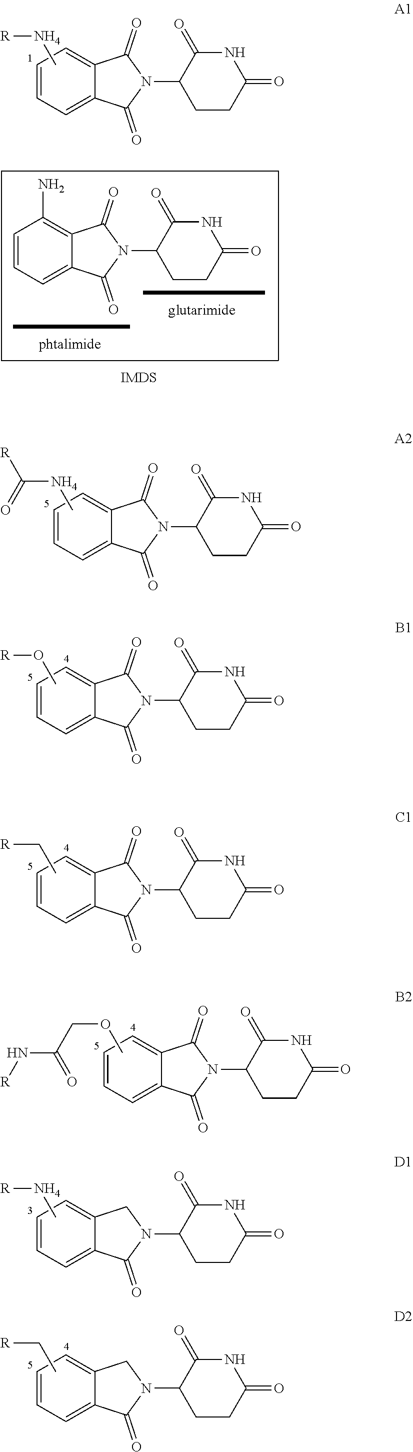

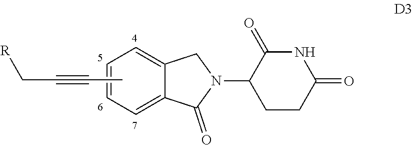

Additional E3 ligase ligands for CRBN include:

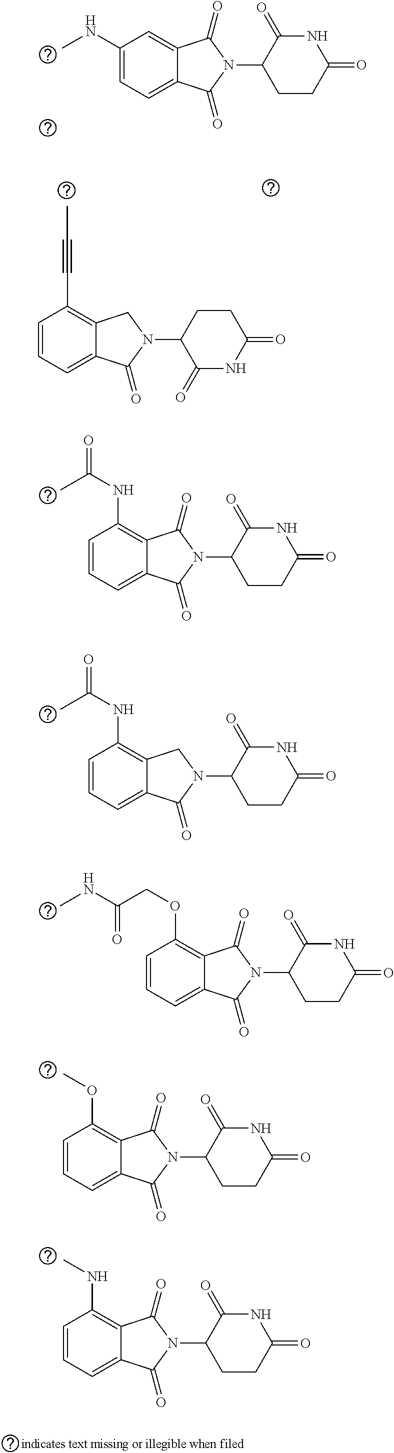

Illustrative E3 ligase ligands for VHL include:

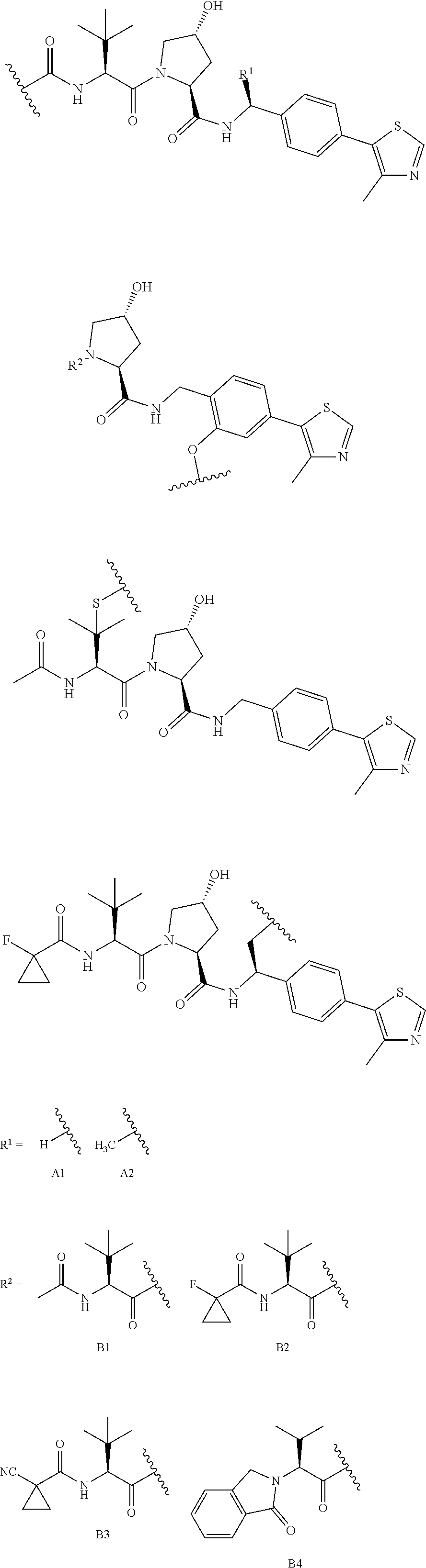

Additional E3 ligase ligands include:

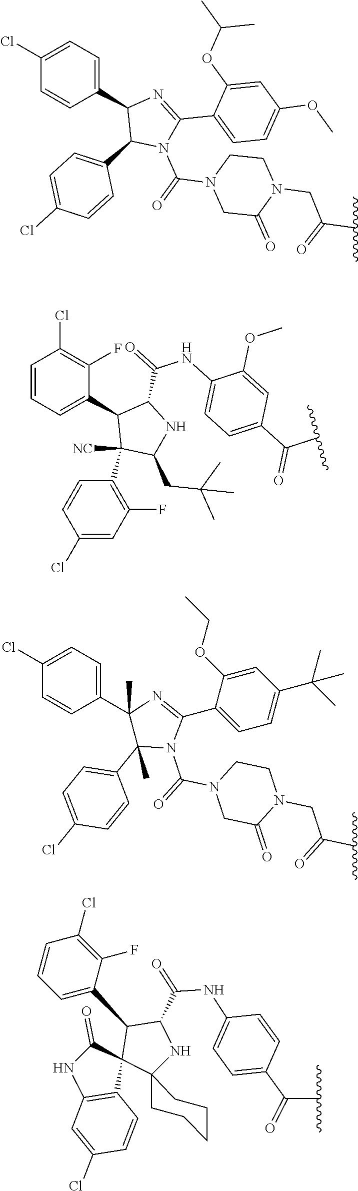 [SMILES: *C(=O)CN1CCN(C(=O)N2C(c3ccc(OC)cc3OC(C)C)=N[C@H](c3ccc(Cl)cc3)[C@@H]2c2ccc(Cl)cc2)CC1=O ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1=O ; *C(=O)c1ccc(NC(=O)[C@@H]2NC3(CCCCC3)[C@@]3(C(=O)Nc4cc(Cl)ccc43)[C@H]2c2cccc(Cl)c2F)cc1]

Illustrative E3 ligase ligands for IAP include:

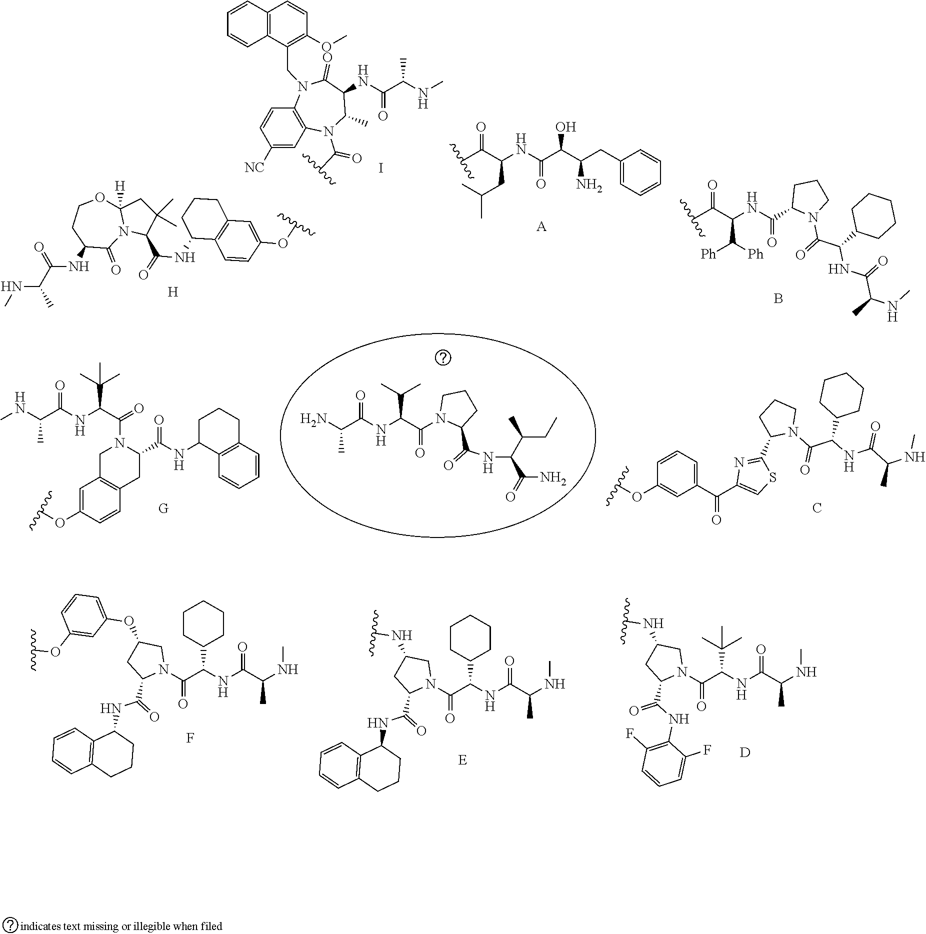

Illustrative E3 ligase ligands for MDM2 include:

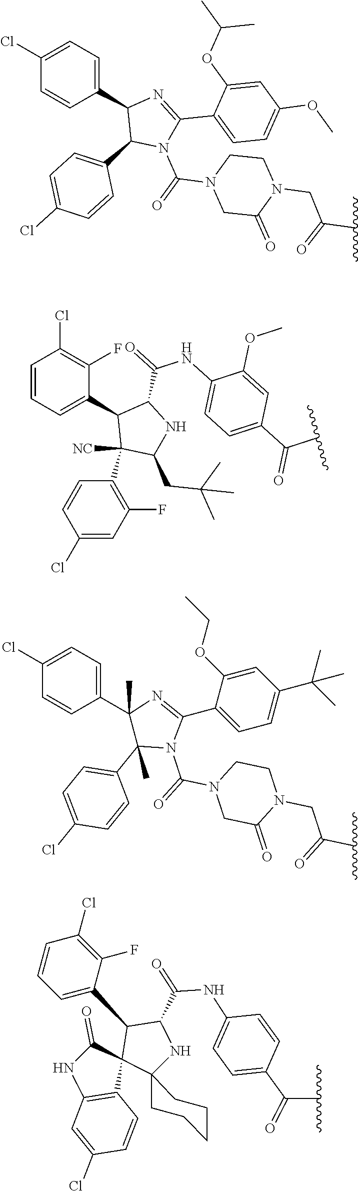 [SMILES: *C(=O)CN1CCN(C(=O)N2C(c3ccc(OC)cc3OC(C)C)=N[C@H](c3ccc(Cl)cc3)[C@@H]2c2ccc(Cl)cc2)CC1=O ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1=O ; *C(=O)c1ccc(NC(=O)[C@@H]2NC3(CCCCC3)[C@@]3(C(=O)Nc4cc(Cl)ccc43)[C@H]2c2cccc(Cl)c2F)cc1]

General structures for E3 ligase ligands include:

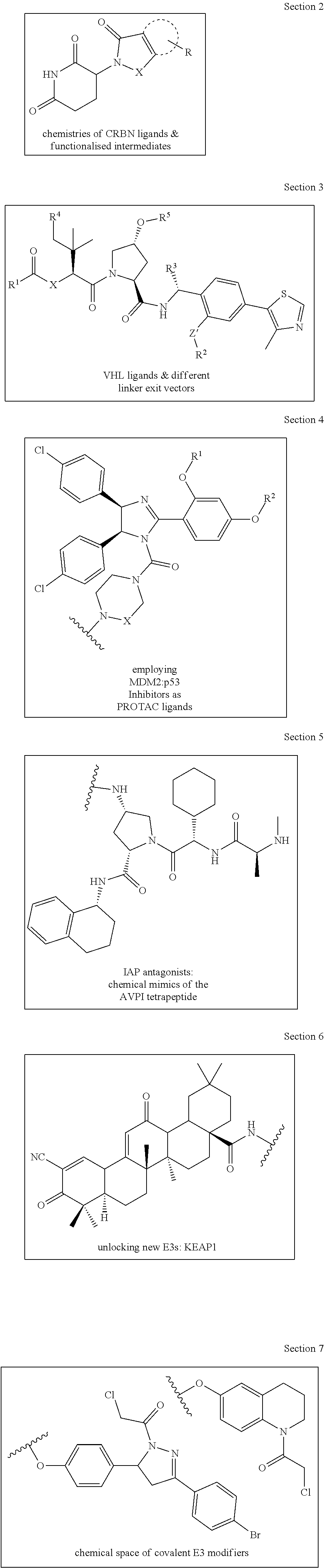

wherein for the CRBN ligand R is thiol, alcohol, alkyne, alkene, benzyl, amino, or amido; and X is H or C═O
wherein for the VHL ligand X is NH, Y is SR, wherein R is H or alkyl; Z is OH; R¹ is NHR, OR, or linker to L, wherein R is H or alkyl; R² is alternative linker to L; R³ is H or CH₃; R⁴ is alternative linker to L; and R⁵ is H or a caging group
wherein for the MDM2 ligand X is C═O or CH₂; R¹ is CH(CH₃)₂ or CH₂CH₃; and R² is CH₃ or an amide linker to L.

The linker L for use with a CRBN ligand include:

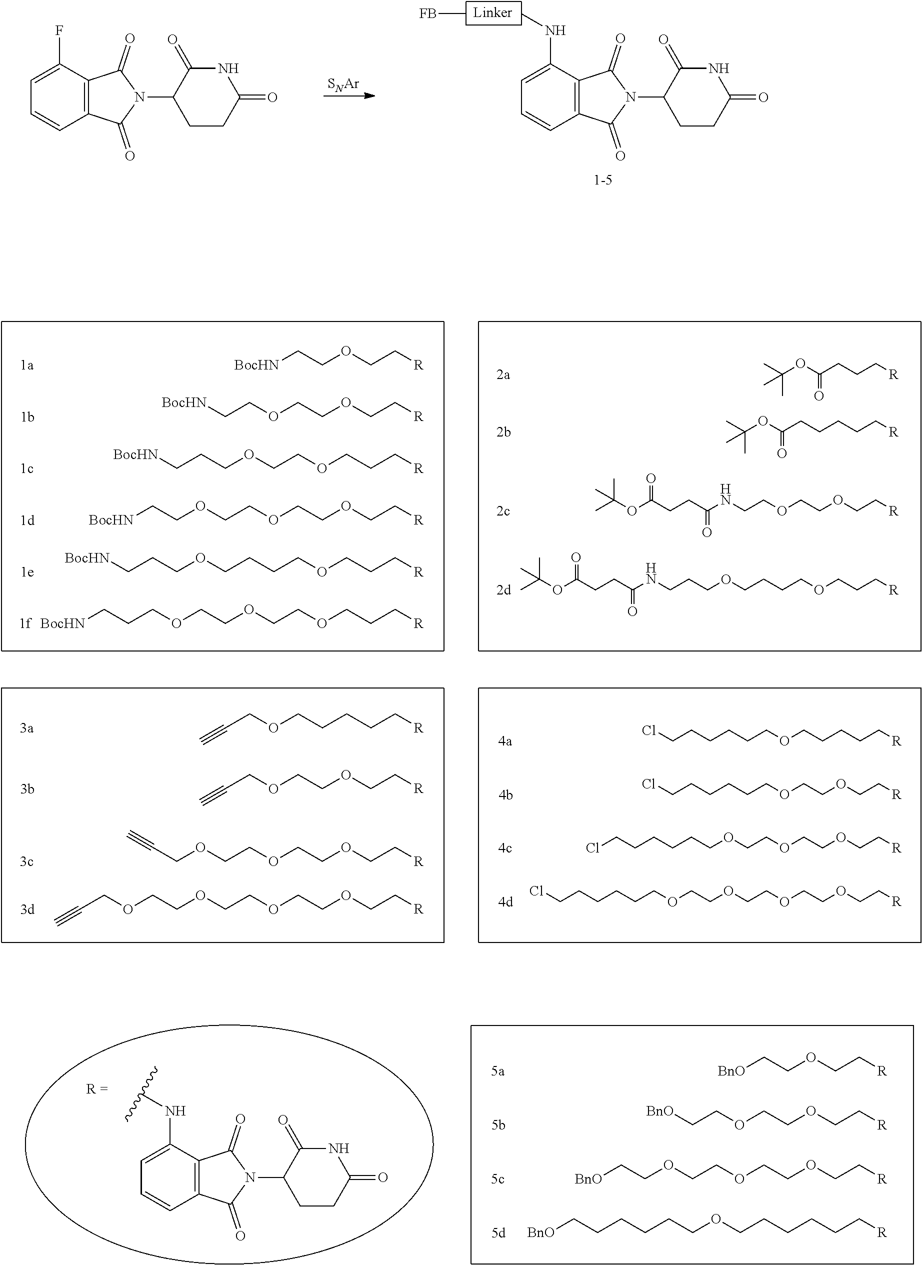

Other linkers (L) include:

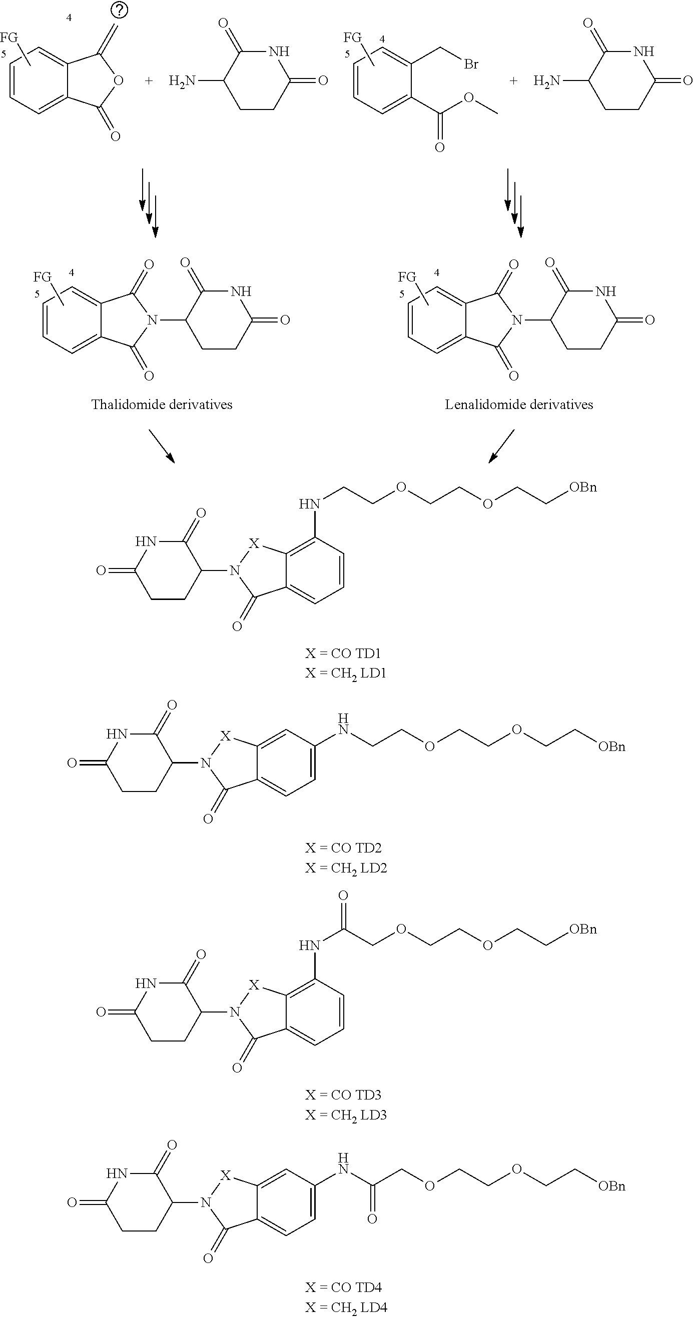

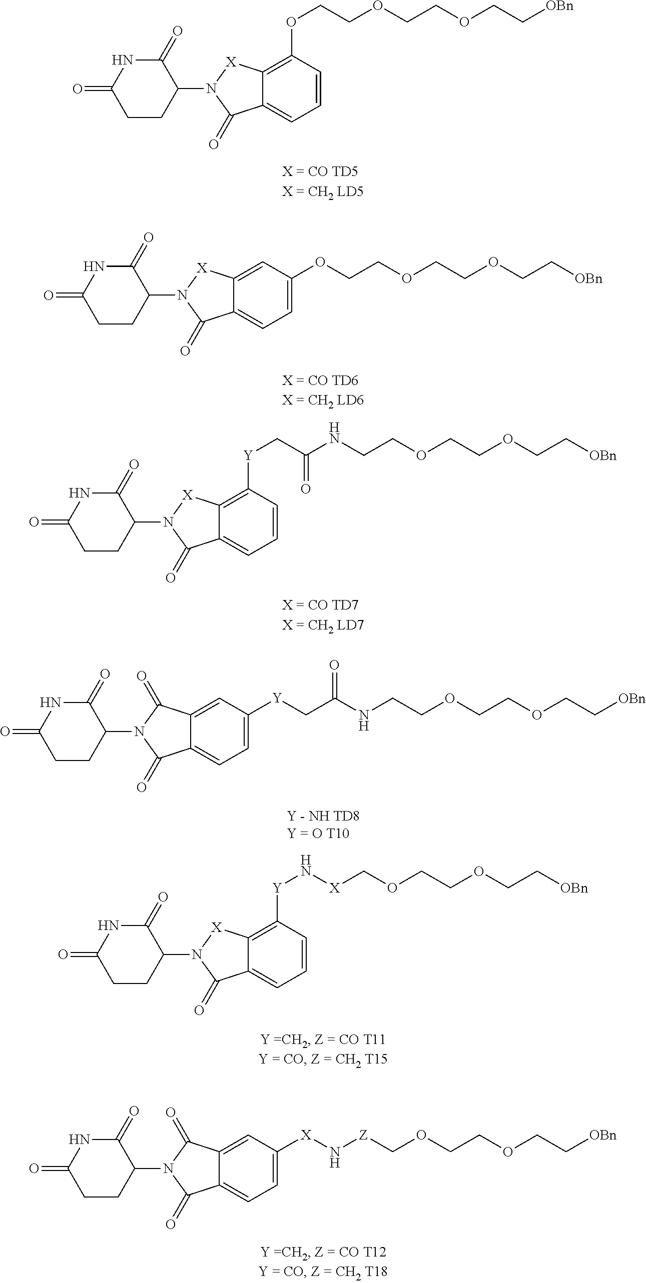

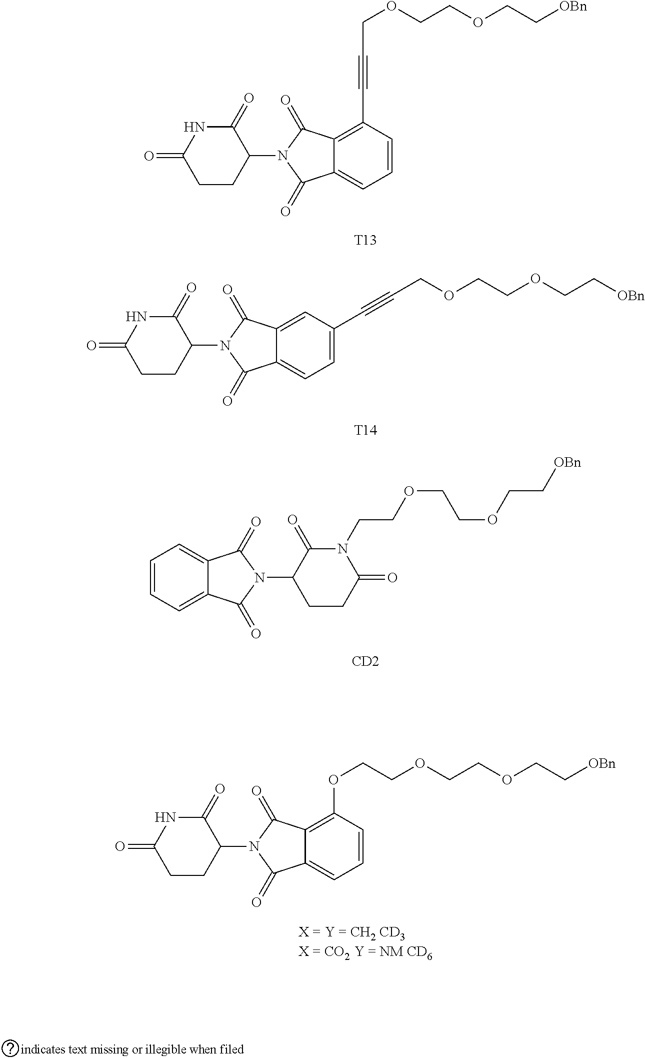

Additional linker (L) groups include:

| Structure | Linker [symbol] |
|---|---|
| 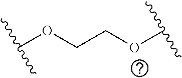 [SMILES: *OCCO*] | PEG |
| 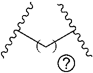 | Allyl [symbol] |
| 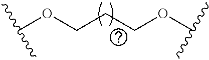 | Other Glycol |
| 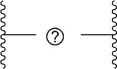 | Alkyne |
| 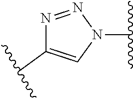 [SMILES: *c1cn(*)nn1] | Tri [symbol] |
| 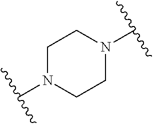 [SMILES: *N1CCN(*)CC1] | Piperazine |
| 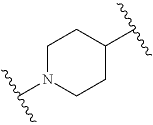 [SMILES: *C1CCN(*)CC1] | Piperidine [symbol] |

[symbol]

indicates data missing or illegible when filed

Illustrative general structures for A in the compound A-L-B for bioconjugation to the respective unnatural amino acid where R is the point of attachment to the remainder of the A-L-B compound are shown below.

For tetrazine-UAA:

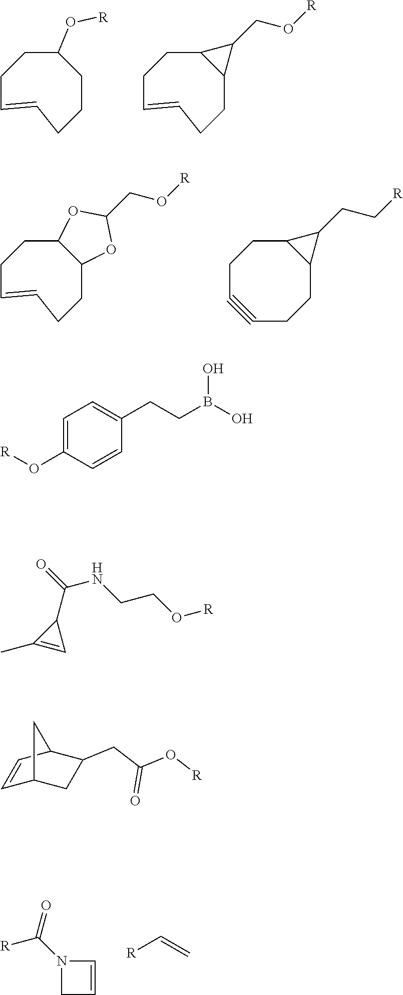

For dienophile-UAA:

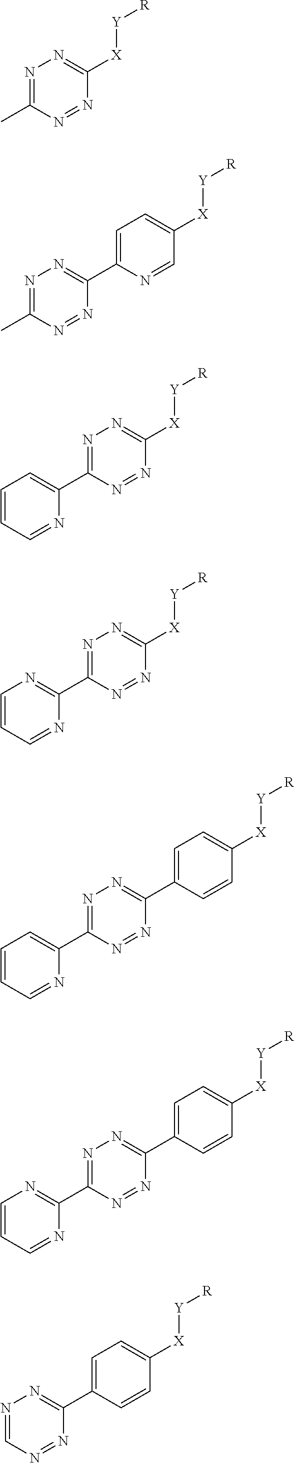

wherein X is CH₂ or C═O; Y is NH; and R is attachment to L.

For azide-UAA:

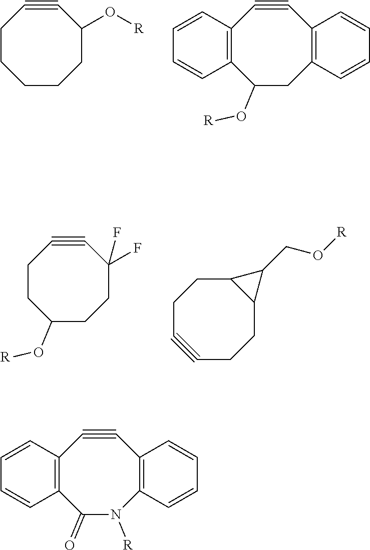

Illustrative bioconjugation ligands A include:

Second-order rate constant (M⁻¹s⁻¹)

| | | | Organic | Mixture | |
|---|---|---|---|---|---|
| | | | solvent | (Org water) | Aqueous solvent |
| Reactivity | a | 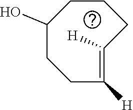 | 1140^{Ref 5} | 2000 (90:10)^{Ref 5} | 22600 (eq iso)^{Ref 3} 80200 (ax iso) |
| | | TCO + T [symbol] | | | |
| | b | 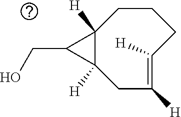 | 22000^{Ref 3} | | 3300000^{Ref 2} |
| | | aTCO + T [symbol] | | | |
| | c | 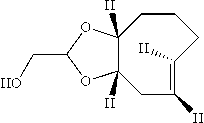 | | 167000 (55:45)^{Ref 3} | 366000 (syn iso)^{Ref 3} 318000(anti iso) |
| | | aTCO + T [symbol] | | | |
| | d | 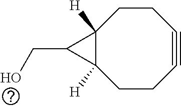 [SMILES: OCC1[C@@H]2CCC#CCC[C@@H]12] | 45^{Ref 4} | 1245 (55:45)^{Ref 6} | |
| | | #CN + T [symbol] | | | |
| | e | 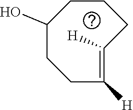 | | 7.5 (50:50)^{Ref 6} | |
| | | TCO + T [symbol] | | | |
| | f | 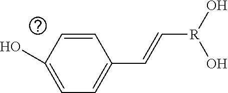 | | | 27^{Ref 7} |
| | | YIM + T [symbol] | | | |
| | g | 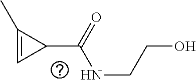 | | 2.3 (50:50)^{Ref 5} | |
| | | 1-methyl-O-R Cyclo [symbol] | | | |
| | h | 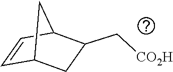 [SMILES: O=C(O)CC1CC2C=CC1C2] | | | 1.9^{Ref 9} |
| | | Norb [symbol] | | | |
| | i | 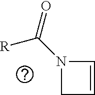 | | 0.39 (12:88)^{Ref 10} | |
| | | N'acyl [symbol] | | | |
| | j | 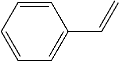 | | 0.05 (50:50)^{Ref 11} | |
| | | S [symbol] | | | |
| | k | 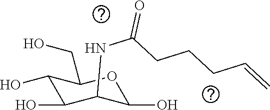 [SMILES: C=CCCCC(=O)NC1C(O)OC(CO)C(O)C1O] | | | 0.021^{Ref 12} |
| | | [symbol] | | | |
| | l | 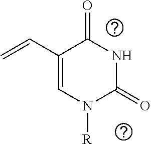 | | 0.021 (67:00)^{Ref 11} | |
| | | V [symbol] | | | |
| | m | 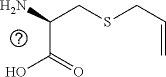 | | 0.002 (50:50)^{Ref 13} | |
| | | S-allyl Cys + T [symbol] | | | |

Dieno precursors

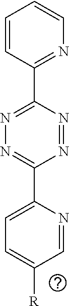

[symbol]

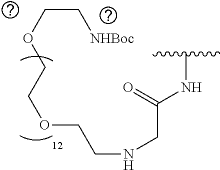

[symbol]

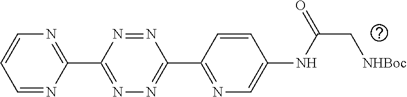 [SMILES: CC(C)(C)OC(=O)NCC(=O)Nc1ccc(-c2nnc(-c3ncccn3)nn2)nc1]

T

[symbol]

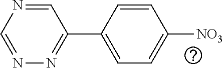

T

[symbol]

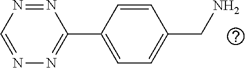 [SMILES: NCc1ccc(-c2nncnn2)cc1]

T

[symbol]

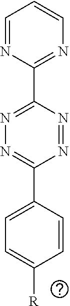

[symbol]

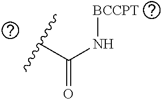

[symbol]

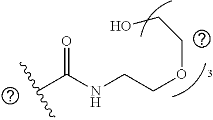

^{Ref 1}JACS, 2008, 130, 13518
^{Ref 2}Chem Sci, 2014, 5, 3770
^{Ref 3}JACS, 2011, 133, 9646
^{Ref 4}Chem Comm, 2012, 48, 1736
^{Ref 5}JACS, 2012, 134, 10317
^{Ref 6}JACS, 2015, 137, 8388
^{Ref 7}ACIE, 2016, 55, 12243
^{Ref 8}Chem Ecr J, 2014, 20, 3365
^{Ref 9}Bioconjug Chem, 2008, 19, 2297
^{Ref 10}Org Lett, 2014, 15, 2744
^{Ref 11}ACIE, 2014, 53, 9168
^{Ref 12}ACIE, 2013, 52, 4265
^{Ref 13}ACIE, 2016, 55, 1468 [symbol]
indicates data missing or illegible when filed

Small molecule ligands for reaction with azide-modified unnatural amino acids include:

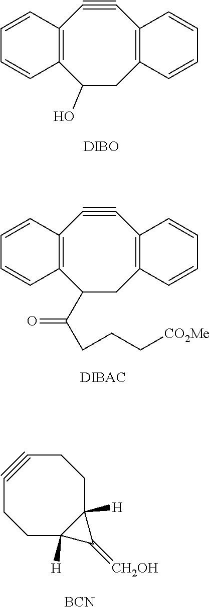

for strain-promoted azide alkyne cycloaddition.

An illustrative A-L-B compound has a structure of:

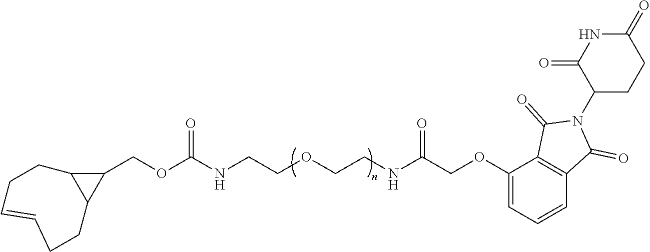

sTCO-PEGₙ-CRBN
wherein n is 2 to 23, more particularly 2 or 9.

Examples of unnatural amino acids that include a bioconjugation handle are shown below.

Illustrative tetrazine-modified unnatural amino acids include:

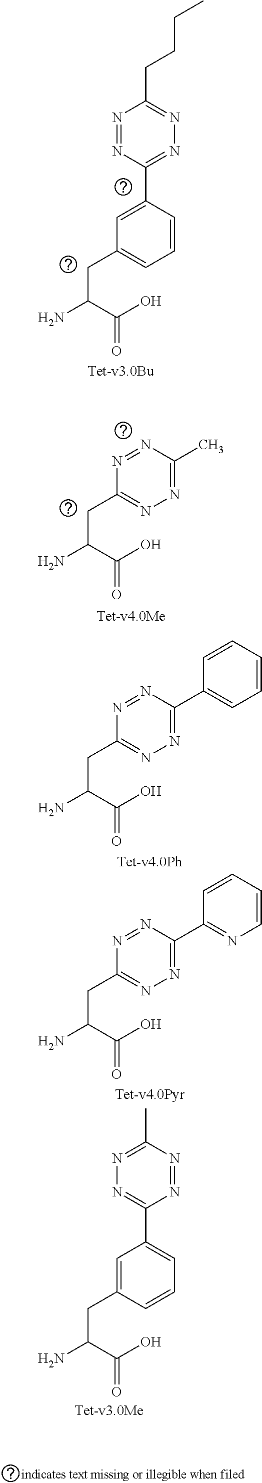

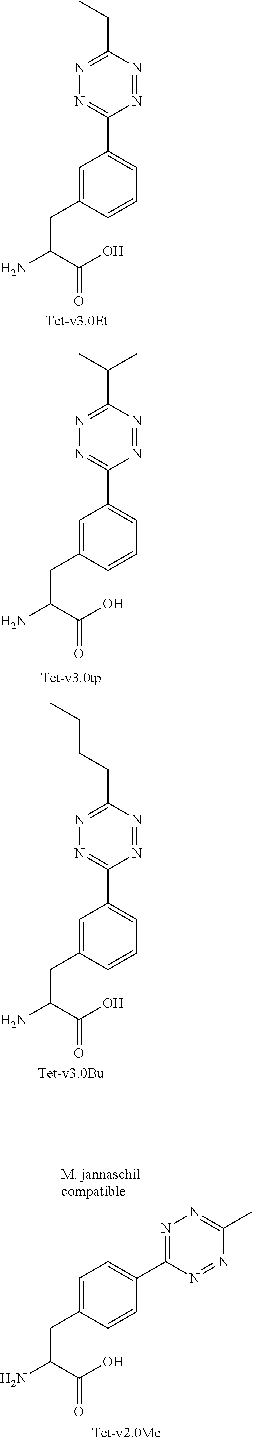 [SMILES: CCc1nnc(-c2cccc(CC(N)C(=O)O)c2)nn1 ; CC(C)c1nnc(-c2cccc(CC(N)C(=O)O)c2)nn1 ; CCCCc1nnc(-c2cccc(CC(N)C(=O)O)c2)nn1 ; Cc1nnc(-c2ccc(CC(N)C(=O)O)cc2)nn1]

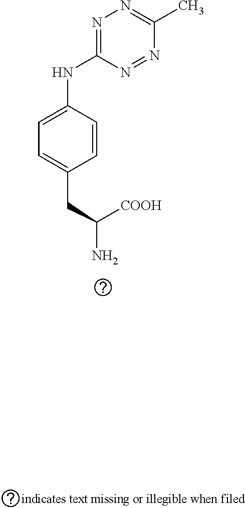 [SMILES: Cc1nnc(Nc2ccc(C[C@H](N)C(=O)O)cc2)nn1]

Additional IEDDA reactive unnatural amino acids include:

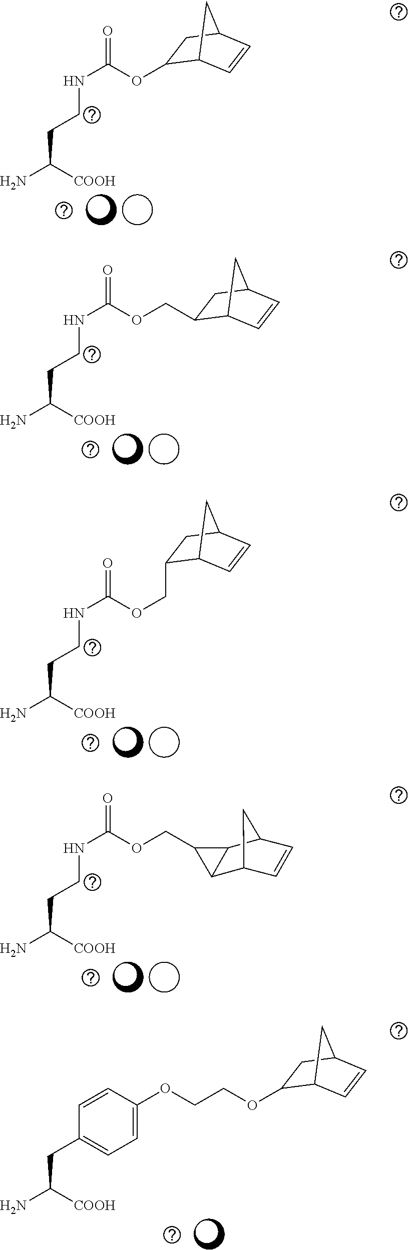 [SMILES: N[C@@H](CCNC(=O)OC1CC2C=CC1C2)C(=O)O ; N[C@@H](CCNC(=O)OCC1CC2C=CC1C2)C(=O)O ; N[C@@H](CCNC(=O)OCC1CC2C=CC1C2)C(=O)O ; N[C@@H](CCNC(=O)OCC1C2C3C=CC(C3)C12)C(=O)O ; N[C@@H](Cc1ccc(OCCOC2CC3C=CC2C3)cc1)C(=O)O]

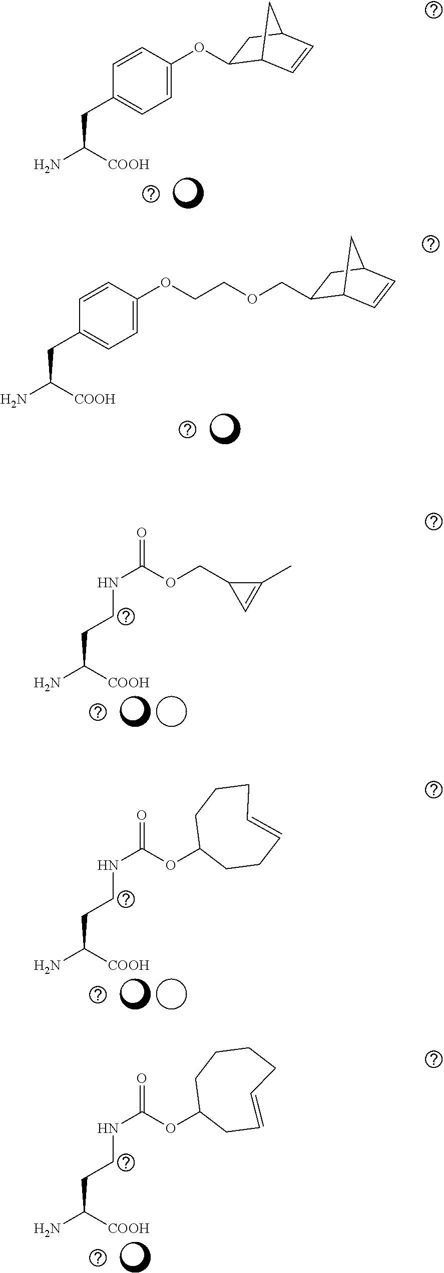 [SMILES: N[C@@H](Cc1ccc(OC2CC3C=CC2C3)cc1)C(=O)O ; N[C@@H](Cc1ccc(OCCOCC2CC3C=CC2C3)cc1)C(=O)O ; CC1=CC1COC(=O)NCC[C@H](N)C(=O)O ; N[C@@H](CCNC(=O)OC1CC/C=C/CCC1)C(=O)O ; N[C@@H](CCNC(=O)OC1C/C=C/CCCC1)C(=O)O]

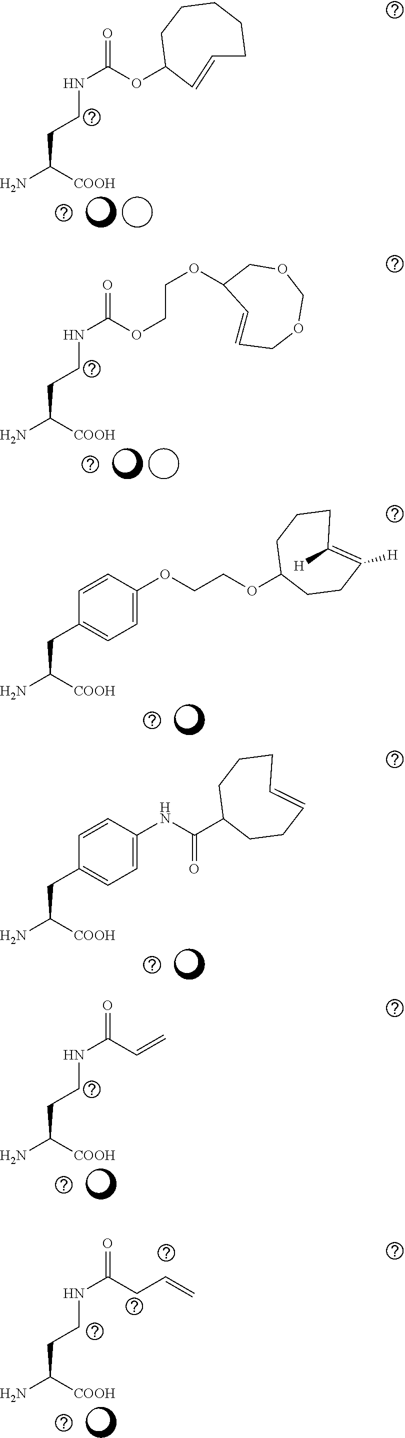

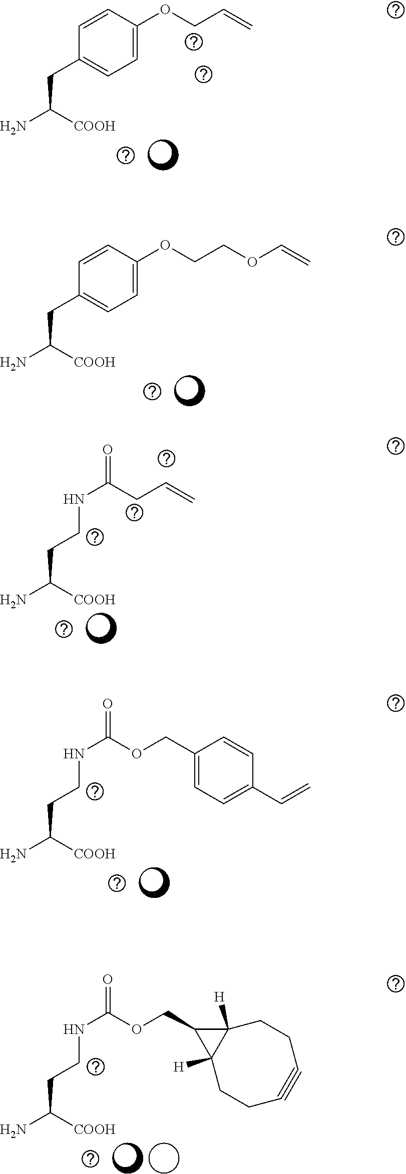 [SMILES: C=CCOc1ccc(C[C@H](N)C(=O)O)cc1 ; C=COCCOc1ccc(C[C@H](N)C(=O)O)cc1 ; C=CCC(=O)NCC[C@H](N)C(=O)O ; C=Cc1ccc(COC(=O)NCC[C@H](N)C(=O)O)cc1 ; N[C@@H](CCNC(=O)OC[C@H]1[C@@H]2CCC#CCC[C@@H]21)C(=O)O]

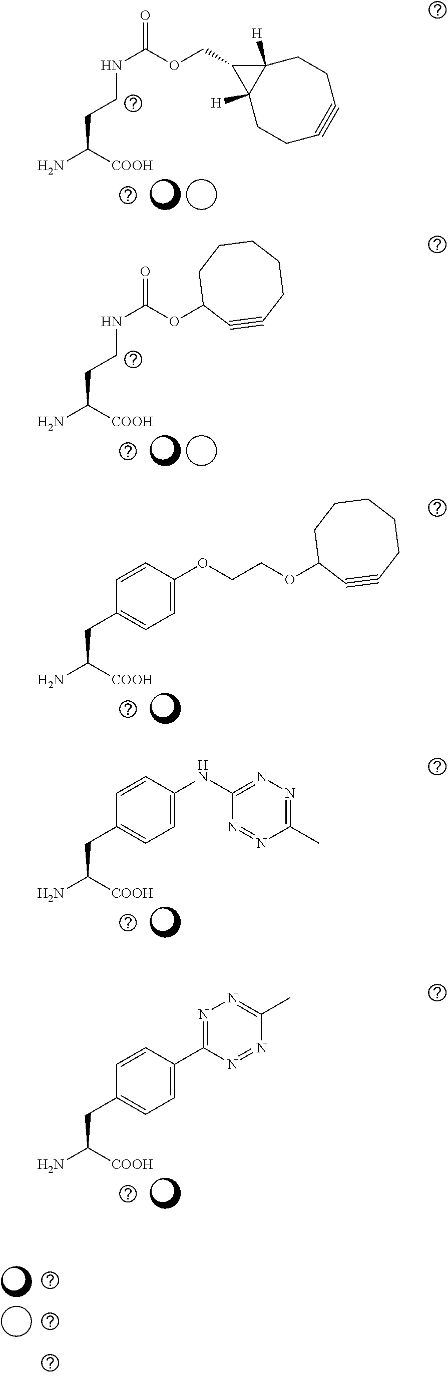 [SMILES: N[C@@H](CCNC(=O)OC[C@@H]1[C@@H]2CCC#CCC[C@@H]21)C(=O)O ; N[C@@H](CCNC(=O)OC1C#CCCCCC1)C(=O)O ; N[C@@H](Cc1ccc(OCCOC2C#CCCCCC2)cc1)C(=O)O ; Cc1nnc(Nc2ccc(C[C@H](N)C(=O)O)cc2)nn1 ; Cc1nnc(-c2ccc(C[C@H](N)C(=O)O)cc2)nn1]

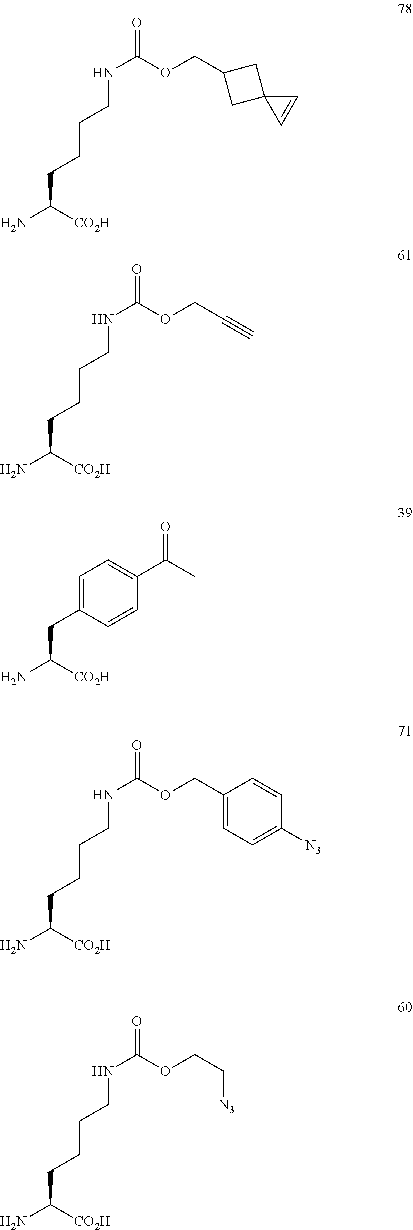 [SMILES: N[C@@H](CCCCNC(=O)OCC1CC2(C=C2)C1)C(=O)O ; C#CCOC(=O)NCCCC[C@H](N)C(=O)O ; CC(=O)c1ccc(C[C@H](N)C(=O)O)cc1 ; [N-]=[N+]=Nc1ccc(COC(=O)NCCCC[C@H](N)C(=O)O)cc1 ; [N-]=[N+]=NCCOC(=O)NCCCC[C@H](N)C(=O)O]

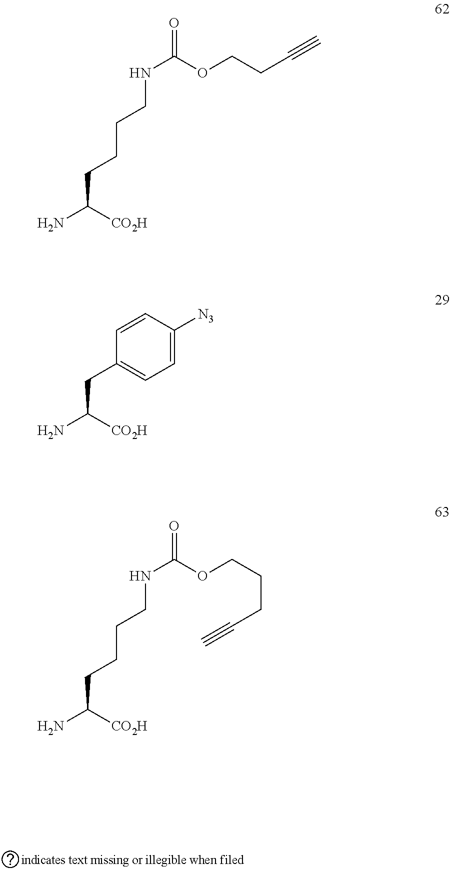 [SMILES: C#CCCOC(=O)NCCCC[C@H](N)C(=O)O ; [N-]=[N+]=Nc1ccc(C[C@H](N)C(=O)O)cc1 ; C#CCCCOC(=O)NCCCC[C@H](N)C(=O)O]

An example of an unnatural amino acid that include a bioconjugation handle has a structure of:

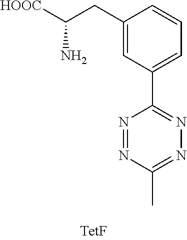 [SMILES: Cc1nnc(-c2cccc(C[C@H](N)C(=O)O)c2)nn1]

Particular examples of the presently disclosed compounds may include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center (C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc.

In one embodiment the bioorthogonal E3 ligase ligand consists of an IEDDA-reactive handle for reaction with its UAA partner displayed on a protein surface, connected to an E3 ligase ligand via a PEG linker. The two E3 ligase ligands commonly used in classical PROTAC design are the thalidomide derivative recruiter of the cereblon (CRBN) E3 ligase and the peptidomimetic ligand known to recruit the Von-Hippel Lindau (VHL) E3 ligase. The results demonstrate that this novel technology can be used to selectively recruit CRBN to the POI without off-target degradation, and to directly compare degradation at different protein surface sites.

For proteins with poorly defined structures or substrate promiscuity, covalent ligands have been employed for PROTAC design. Covalent PROTACs utilize targeted covalent inhibitors, or a ligand specific to a POI binding domain that is modified with an electrophilic moiety for reaction with a nearby nucleophilic residue. The irreversible nature of covalent PROTACs counteracts the event-driven pharmacology and resultant potency of non-covalent PROTACs, but covalency benefits from improved ligand efficiency and selectivity, decreased toxicity, longer target engagement, and a resistance to target mutations. A bioorthogonal E3 ligase ligand approach broadens the applicability of current covalent PROTACs to any POI that can be modified with a UAA.

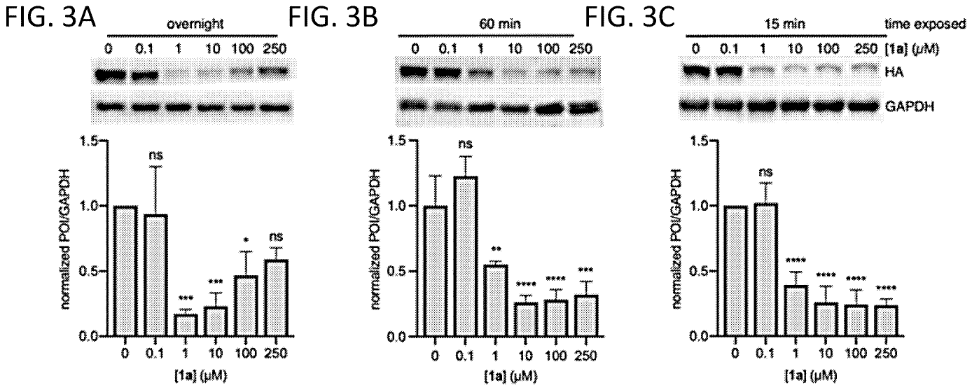

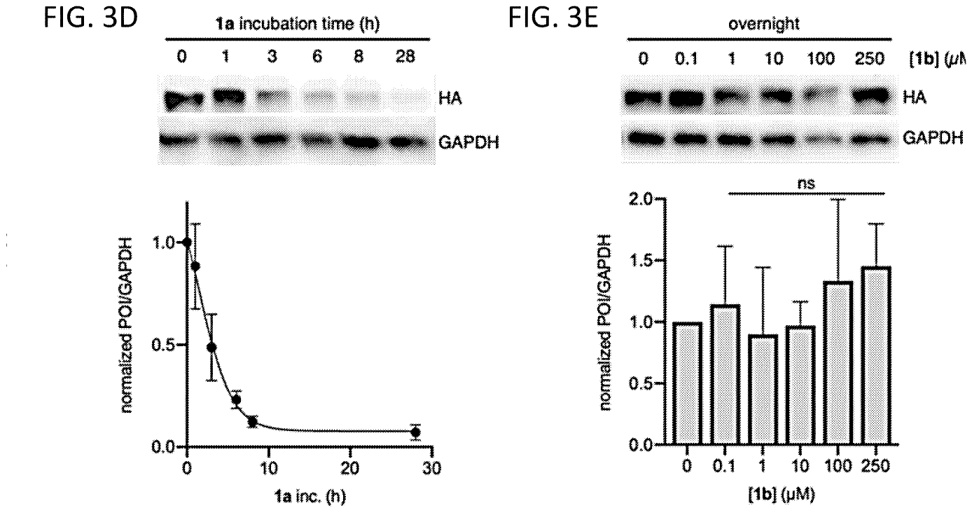

Utilizing the reporter mCherry-EGFP-Y151TetF, conditions were optimized for protein degradation by the novel bioorthogonal E3 ligase ligand disclosed herein, consisting of a strained trans-cyclooctene (sTCO) linked via a PEG linker (n=2 or 9) to the CRBN ligand (sTCO-PEGₙ-CRBN, FIG. 2D). To first establish an optimal concentration of sTCO-PEG₂-CRBN, cells expressing mCherry-EGFP-Y151-HA were incubated with increasing concentrations of compound, keeping the compound-containing media on the cells for the entirety of the experiment. For sTCO-PEG₂-CRBN, optimal degradation was observed between 1 and 10 μM (FIG. 3A). Further, incubation with sTCO-PEG₂-CRBN in the presence of proteasome inhibitors prevented degradation of mCherry-EGFP-Y151TetF, validating that the observed degradation is mediated by the proteasome. Unsurprisingly, a distinct hook effect was observed at higher concentrations (100 to 250 μM) of sTCO-PEG₂-CRBN, indicating that the native proteasomal machinery is saturated by the persistent CRBN ligand and thus unable to induce degradation of the conjugated protein (FIG. 3A).

In an attempt to combat the observed hook effect resultant of high sTCO-PEG₂-CRBN concentrations, the time was limited in which cells were directly exposed to the compound. Specifically, the transfected cells were compound-treated for the indicated time prior to replacement with compound-free media, and overnight incubation. By reducing the total time exposed to sTCO-PEG₂-CRBN from overnight to just 15 or 60 minutes, a distinct decrease was observed in the previously observed hook effect (FIG. 3B-3C). While degradation was still optimal with overnight treatment with 1 or 10 μM of sTCO-PEG₂-CRBN, the observed trend in degradation with decreased exposure times indicated that the bioconjugation reaction was fast, leaving the UPS-mediated degradation as the limiting factor. The kinetics of the degradation were further validated by a time-course, in which cells expressing the TetF reporter were incubated with 10 μM of sTCO-PEG₂-CRBN for the indicated times (FIG. 3D). Substantial degradation was observed within just 6 hours, with near complete knockdown achieved after overnight incubation. To account for variations in TetF-bearing proteins expressed in cells, subsequent degradation experiments were performed with overnight exposure to 10 μM of sTCO-PEG₂-CRBN.

Following successful degradation with sTCO-PEG₂-CRBN, the degradation efficiency of a bioorthogonal E3 ligase ligand bearing a markedly longer PEG-linker was evaluated, as linker length is known to affect PROTAC potency and efficiency. To study the length-activity relationship, sTCO-PEG₉-CRBN (X) was generated, that interestingly did not induce any significant degradation of the reporter (FIG. 3E). This lack of activity is likely due to the spatial orientation of the resultant ternary complex, as the PEG₉ linker is too long to induce ubiquitination of this target. While the sTCO-PEG₉-CRBN did not exhibit degradation of mCherry-EGFP-Y151TetF, the longer linker might yield improved degradation of different sites on the same reporter or for other protein targets.

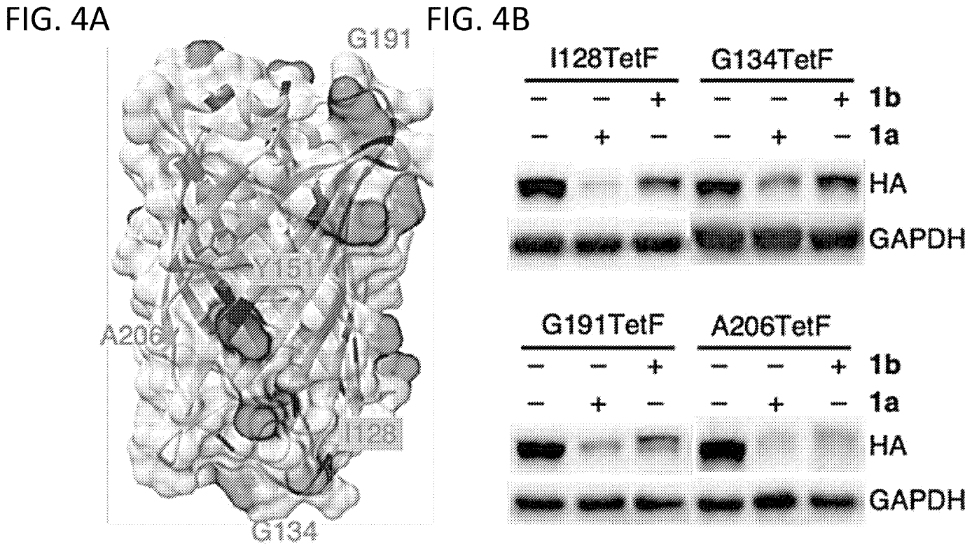

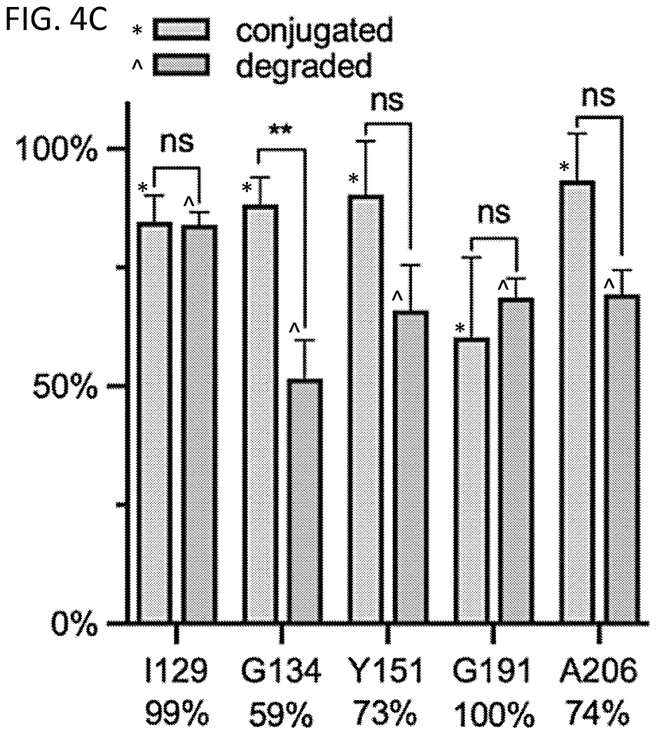

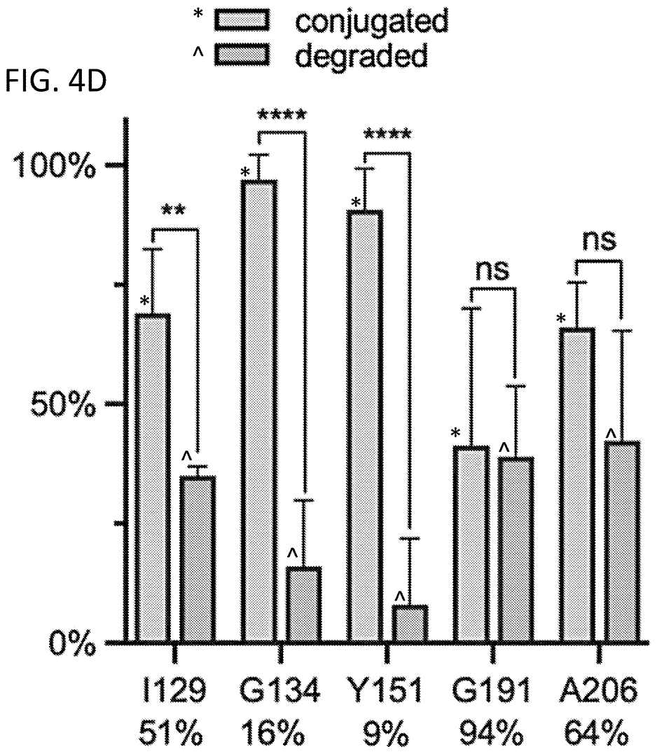

A distinct advantage of the bioorthogonal approach to targeted protein degradation is the ability to directly compare degradation at different locations across a protein surface. This is also particularly useful for the analysis of proteins without known small molecule ligands. As a fluorescent reporter, EGFP has no known small molecule ligand. Thus, with the system optimized to degrade mCherry-EGFP-Y151TetF-HA, four additional EGFP mutants were selected, I128TetF, G134TetF, G191TetF and A206TetF (FIG. 4A), to determine if different sites of ligand recruitment led to varying levels of degradation.

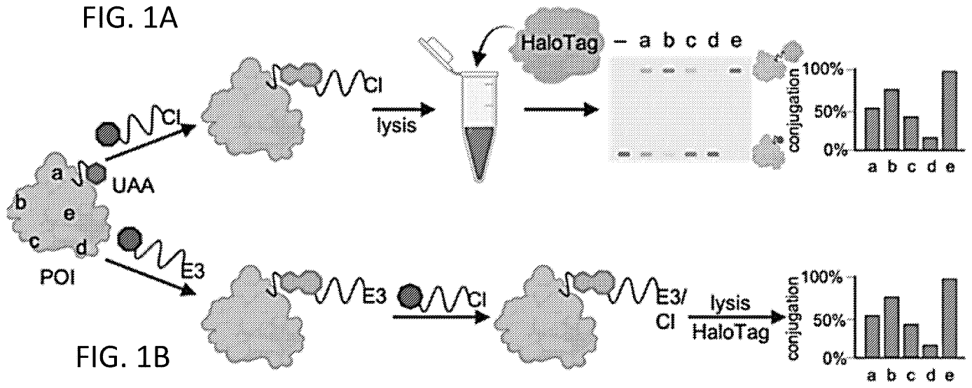

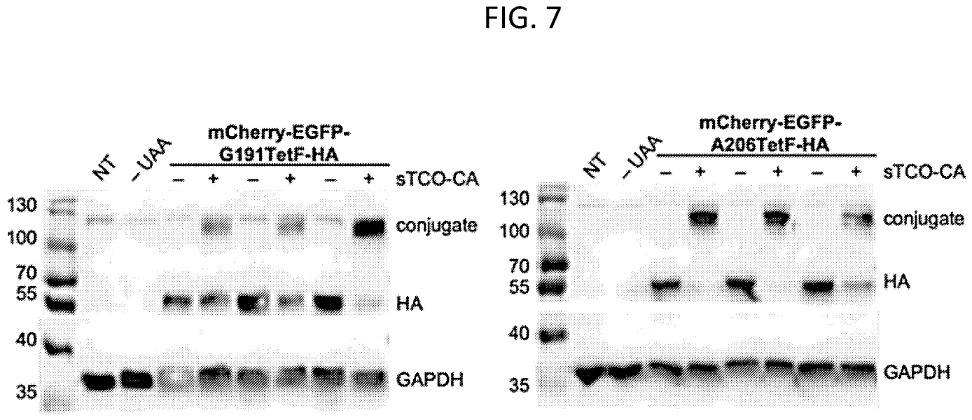

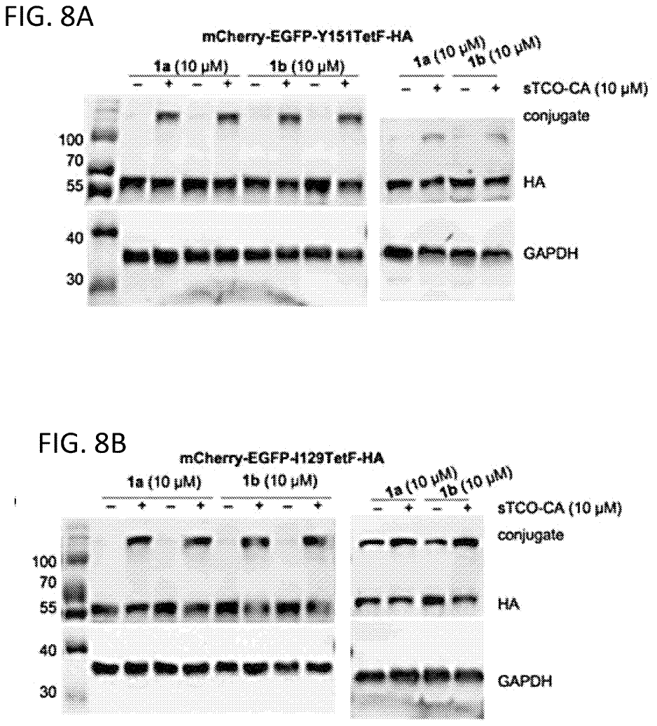

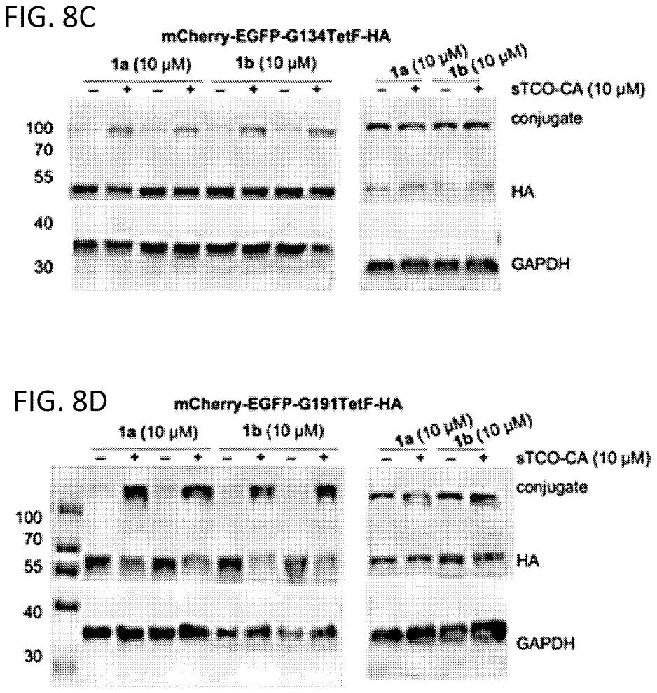

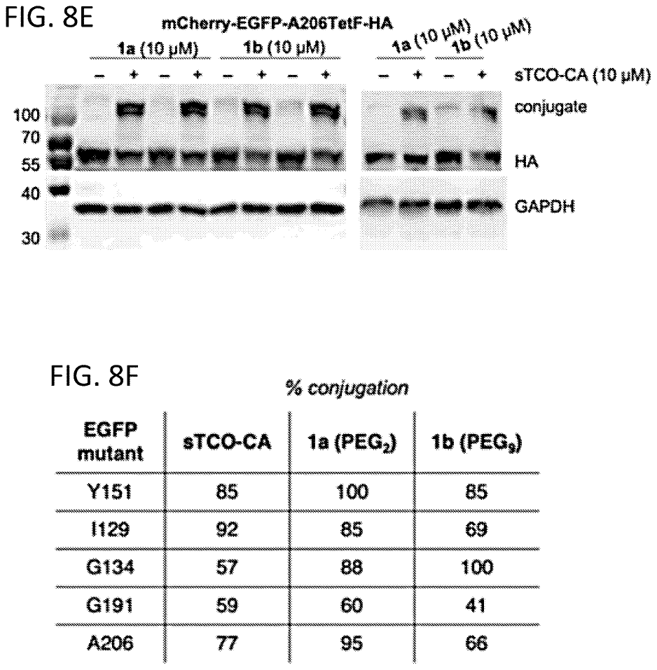

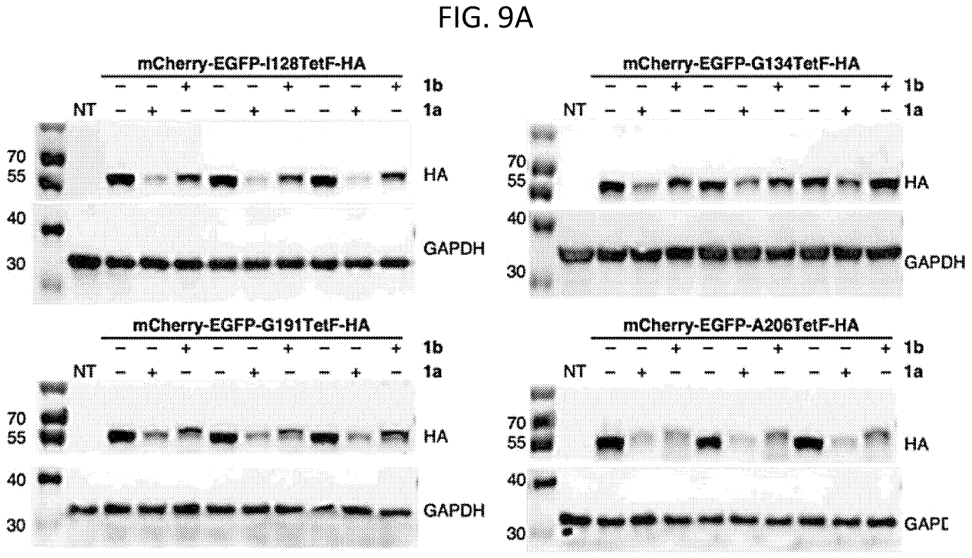

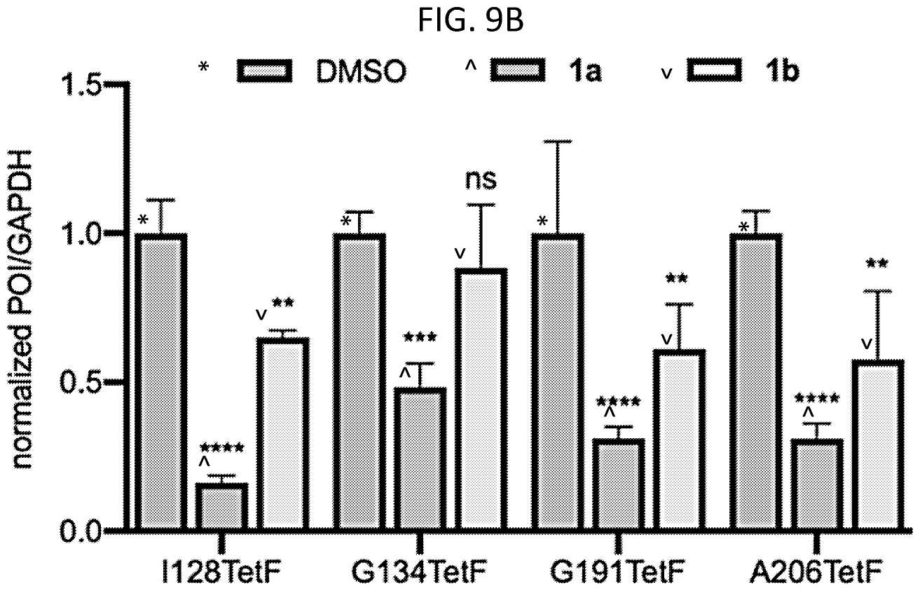

Before comparing the degradation of different mCherry-EGFP mutants, validating the extent of bioconjugation to TetF at each position was required. A method for quantifying in cellulo protein bioconjugation that utilizes a strained trans-cyclooctene chloroalkane (sTCO-CA) to react with TetF and subsequently dimerize with the self-labeling enzyme, HaloTag, was previously depended. Using this technique, the extent of bioconjugation mCherry-EGFP-I129TetF (~90%) and mCherry-EGFP-G134TetF (~55%, FIG. 4D) was quantified. Bioconjugation to two new mCherry-EGFP-TetF mutants using the same method was also quantified (FIG. 7). To more accurately determine the bioconjugation efficiency of sTCO-PEG₂-CRBN and sTCO-PEG₉-CRBN, we utilized a pulse-chase experiment (FIG. 1), and identified respective labeling efficiencies of sTCO-PEG₂-CRBN and sTCO-PEG₉-CRBN to be 85% and 69% (I129TetF), 88% and 100% (G134TetF), 60% and 41% (G191TetF), and 95% and 66% (A206TetF, FIG. 8). By determining the extent of bioconjugation to TetF mutants across the surface of mCherry-EGFP, variations in degradation due to incomplete bioconjugation could be evaluated. For all five mCherry-EGFP-TetF-HA mutants examined, with the exception of G134TetF, nearly every protein that was reacted with sTCO-PEG₂-CRBN was degraded (FIG. 4C). Interestingly, less efficient degradation was observed with sTCO-PEG₉-CRBN (FIG. 4D), which is likely due to the increased linker length preventing the recruited ligase from properly orienting with the POI to induce ubiquitination. The relatively small size and abundance of surface lysine residues made EGFP an ideal target for early optimization of this technique, which were then applied to additional, more diverse protein targets for further investigation into the minutiae of PROTAC design.

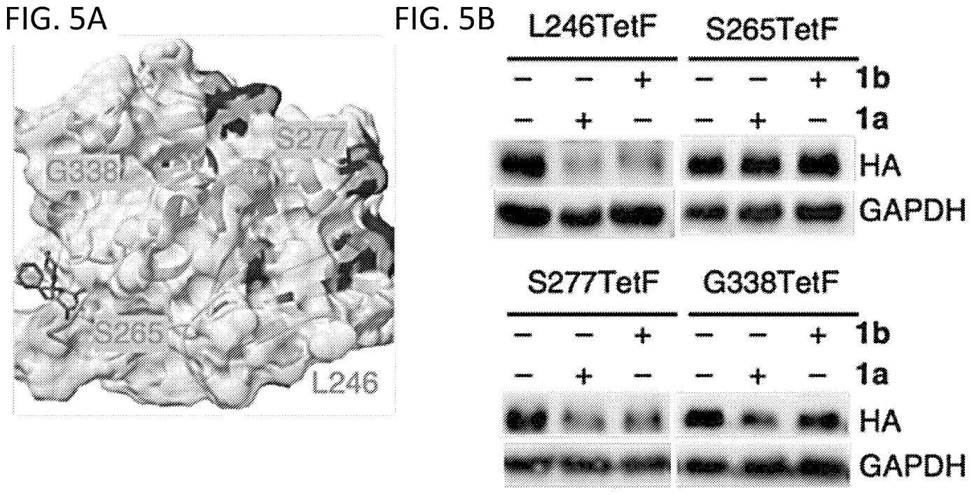

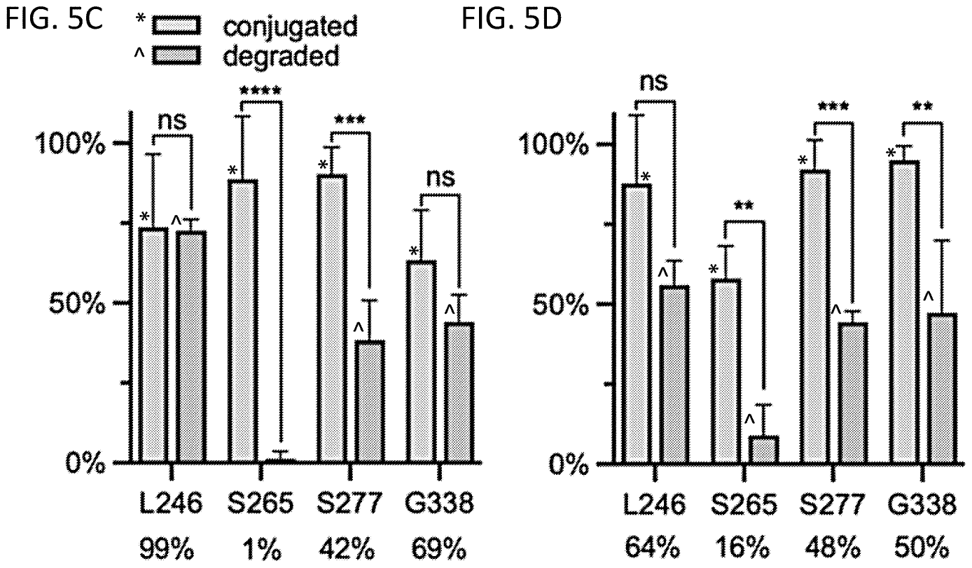

To investigate the spatial requirement of surface lysine residues for degradation efficiency, another small protein was selected without a known PROTAC that has a varied landscape of lysine residues, the mitogen-activated protein kinase dual specificity phosphatase 3 (MKP3, FIG. 5A). MKP3 is involved in the Ras/Raf/MAPK pathway, in which it dephosphorylates MAP kinases ERK, p38, and JNK. There exists a high degree of conservation between structures of MKP3 and other dual specificity phosphatases, which has hindered the discovery of small molecule ligands of this enzyme family. For example, MKP3 (also known as dual specificity phosphatase 6, or DUSP6) has one known allosteric inhibitor, BCI, however this ligand also recognizes and inhibits DUSP1. It is likely that due to this limited specificity of the BCI inhibitor, MKP3 has yet to become a target for PROTAC-mediated degradation. Because MKP3 plays critical roles in signaling processes that affect development, differentiation, and proliferation, it was selected as a model for the application of the bioorthogonal E3 ligase ligands for probing the previously undruggable and “un-PROTAC-able” protein.

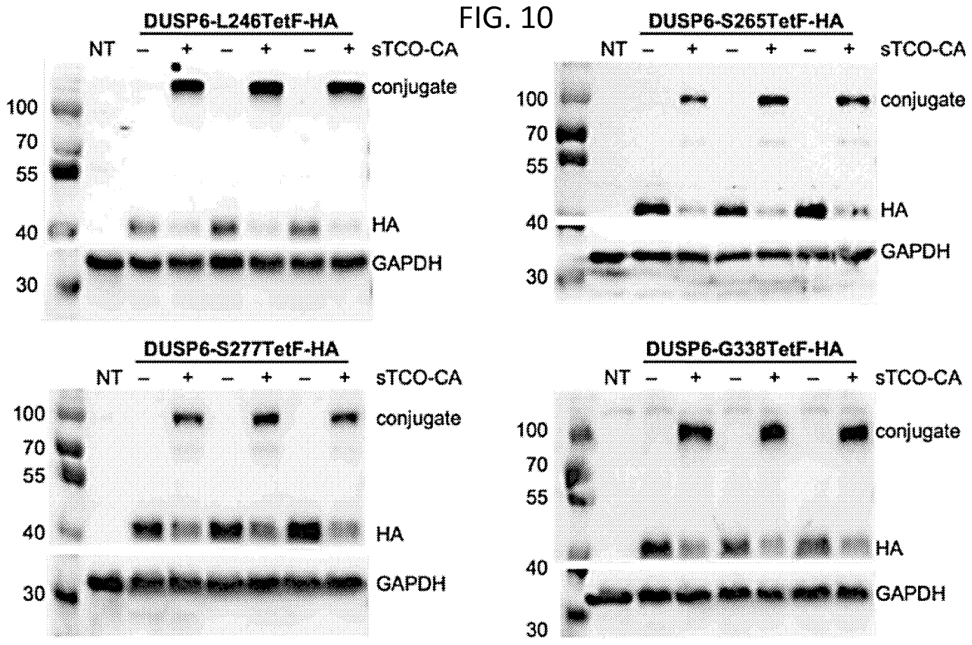

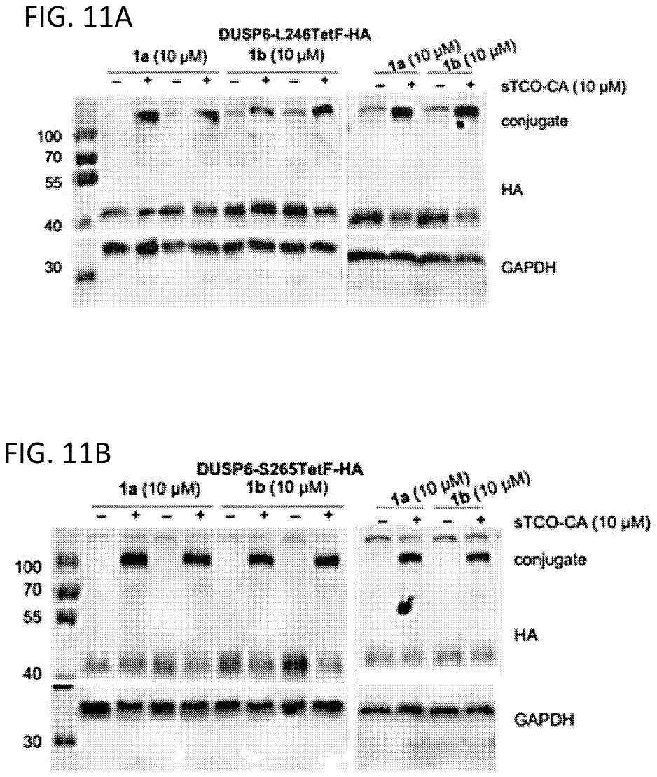

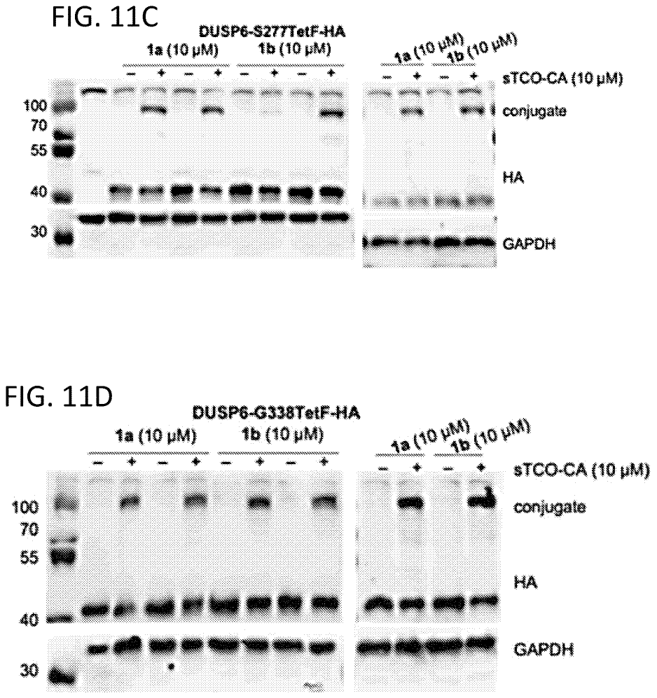

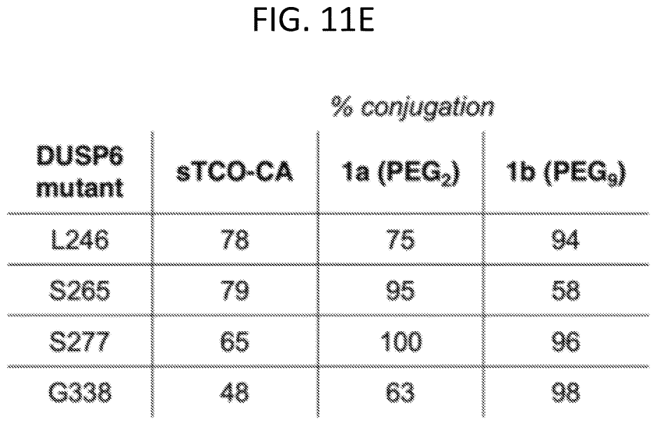

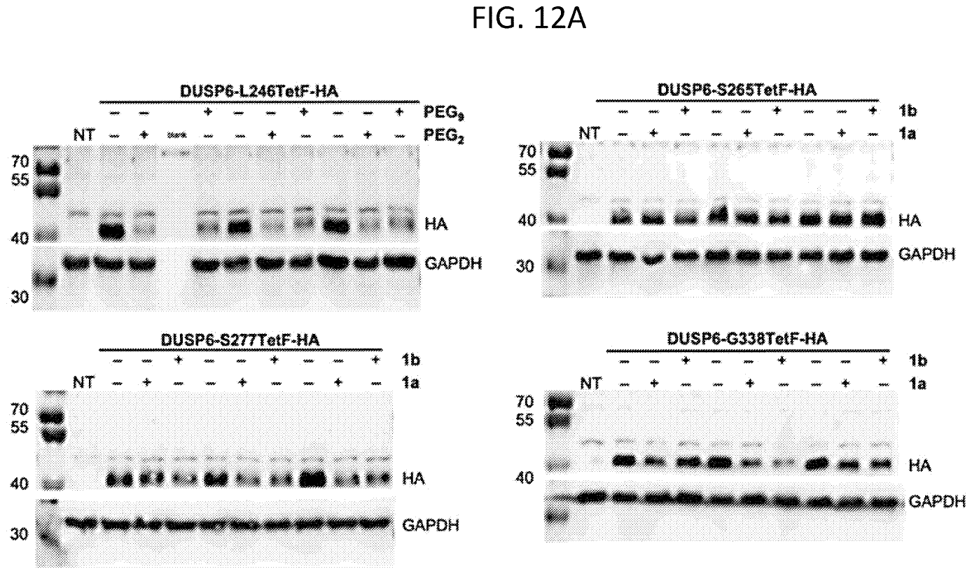

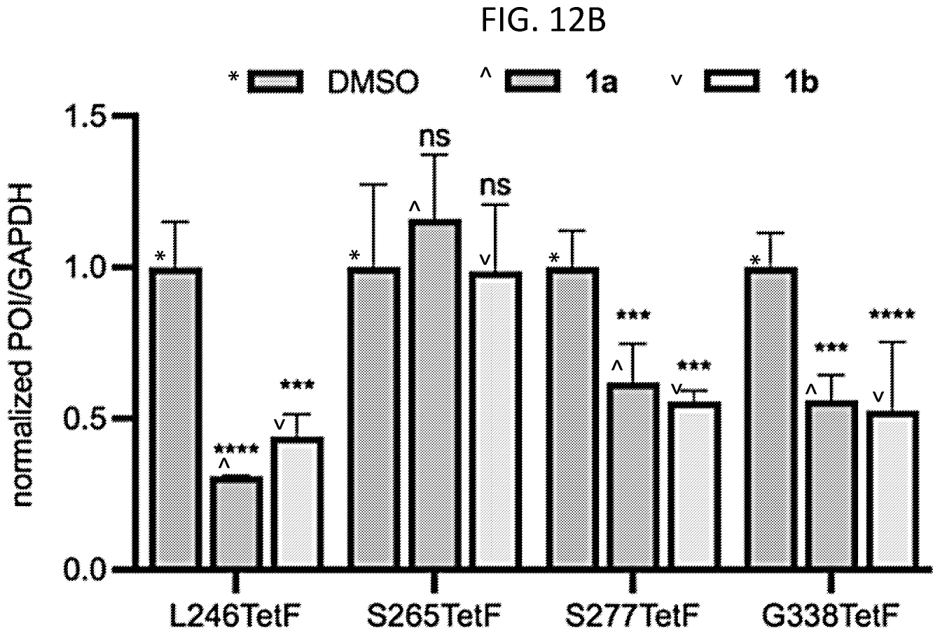

To first test the ability to bioconjugate MKP3, four mutants were generated for which genetic code expansion could incorporate TetF at a surface residue. Based on the crystal structure and location of surface lysine residues, we selected two mutants, L246TetF and S277TetF, for their proximity to lysines, and two mutants positioned away from any lysine residues, S265TetF and G338TetF (FIG. 5A). As protein ubiquitination via the UPS is known to require a proximal lysine, the mutants L246TetF and S277TetF should demonstrate improved degradation efficiency relative to S265TetF or G338TetF. Further, the mutants S265TetF and G338TetF are positioned near the binding site of known MKP3 inhibitor BCI, so these mutants will inform the PROTAC-ability of BCI for MKP3 degradation. Bioconjugation to each MKP3-TetF mutant was quantified utilizing sTCO-CA (FIG. 10), sTCO-PEG₂-CRBN (FIG. 11), and sTCO-PEG₂-CRBN (FIG. 11).

To analyze degradation efficiency, these four TetF-bearing proteins were expressed in HEK293T cells. Utilizing the optimized conditions for EGFP degradation by sTCO-PEGₙ-CRBN, the degradation of the MKP3 mutants was compared. The mutant MKP3-L246TetF was efficiently degraded by both sTCO-PEG₂-CRBN and sTCO-PEG₉-CRBN (FIG. 5B-D). As the incorporated tetrazine is situated near 2-3 surface lysine residues, the observed degradation aligns with the hypothesis that proximal lysine residues are critical for degradation. It is likely that the increased number of lysines within range of the conjugated E3 ligase allowed for the excellent knockdown of MKP3-L246TetF relative to the observed bioconjugation to the same mutant (FIG. 5D). The other mutant located in a lysine-dense region, S277TetF, showed degradation equivalent to the extent of conjugation at the same position (FIG. 5D), meaning that every protein labeled with sTCO-PEGₙ-CRBN was degraded by the proteasome, but the degradation was limited by bioconjugation efficiency. This observed trend in decreased binding could be due to the increased number of charged residues in the surrounding microenvironment, limiting the accessibility of the ligand to the TetF. The poor conjugation to S277TetF limits the degradability of the mutant, but the efficiency of degradation relative to conjugation indicates that this region would still be desirable for E3 ligase recruitment via classical PROTACs. Interestingly, sTCO-PEG₂-CRBN led to moderate degradation of the mutant MKP3-G338TetF despite its relatively poor bioconjugation efficiency and positioning away from lysine (FIG. 5B-4D). The overall small size of MKP3 (44 kDa) allowed for any recruited E3 ligase to orient such that ubiquitin transfer to a distant lysine, relatively “far” from G338TetF was possible, thereby allowing for degradation of this mutant. Finally, the mutant furthest from any surface lysine, MKP3-S265TetF, was not degraded at all by either sTCO-PEGₙ-CRBN ligand, despite a confirmed high efficiency for bioconjugation (FIG. 5B-D). Based on the poor rate of degradation through recruitment of CRBN to MKP3 mutants S265TetF and G338TetF, BCI, even if modified to improve its promiscuity for other DUSPs, would not be useful in the design of a potent PROTAC because it binds too far from necessary lysine residues. Instead, it was demonstrated that MKP3 can be excellently degraded through the recruitment of a bioorthogonally modified CRBN ligand to a lysine-rich region of the previously “un-PROTAC-able” target, ultimately highlighting a different region of MKP3 for ligand design. These results provide an exciting foundation for further investigation into MKP3 and yet undiscovered small molecule ligands for future PROTAC-mediated degradation.

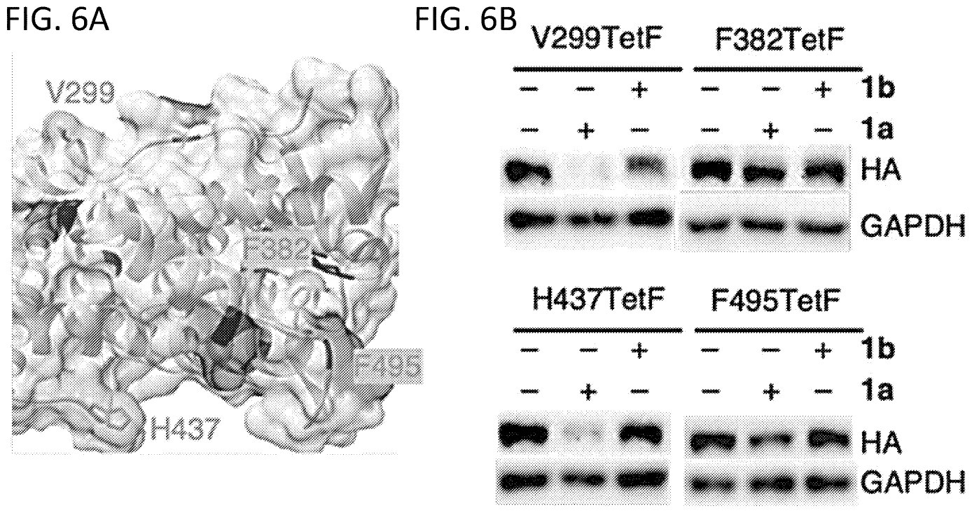

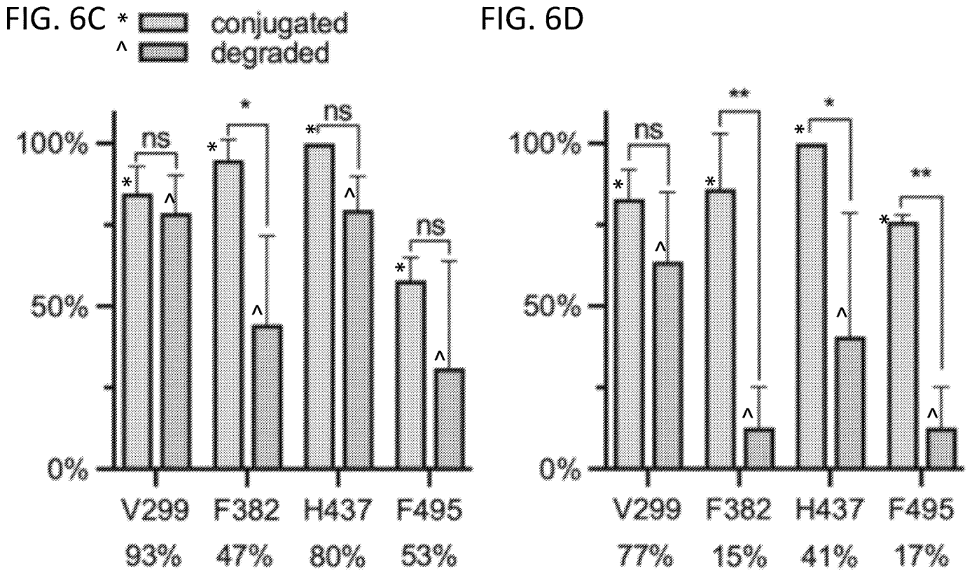

In addition to protein targets without known ligands, the bioorthogonal E3 ligase ligand on target with a known PROTAC was tested. The estrogen related receptor alpha (ERRα) is an orphan nuclear hormone receptor that has been the target of a PROTAC since 2015. The ERRα PROTAC consists of a diaryl-ether thiazolidenedione ligand selective for ERRα (FIG. 6A, green structure) linked via a short hydrocarbon to the peptidomimetic ligand that recruits the VHL E3 ligase. The ERRα ligand of the PROTAC undergoes a reversible covalent reaction with ERRα that leads to high selectivity and potent degradation (1 μM) of ERRα. We aimed to investigate if proteasomal degradation of ERRα could be improved by (A) recruiting a different E3 ligase, (B) recruiting the degrader to a different region of the protein, or (C) varying the length of the PEG linker between E3 ligase and POI ligands.

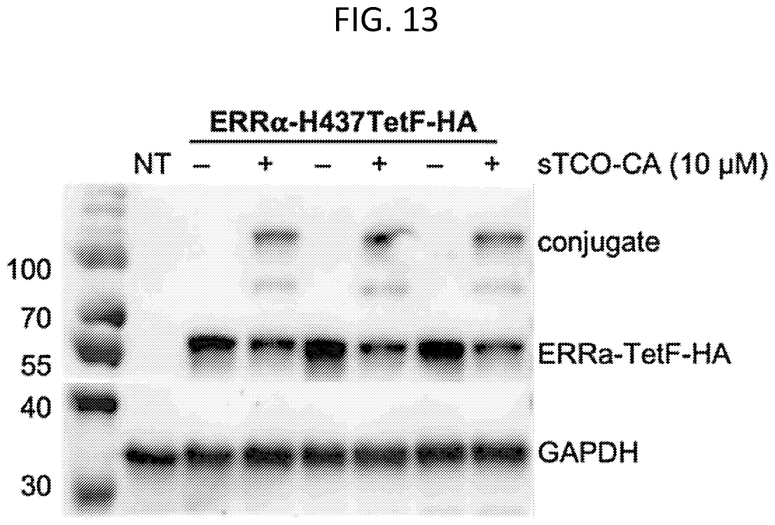

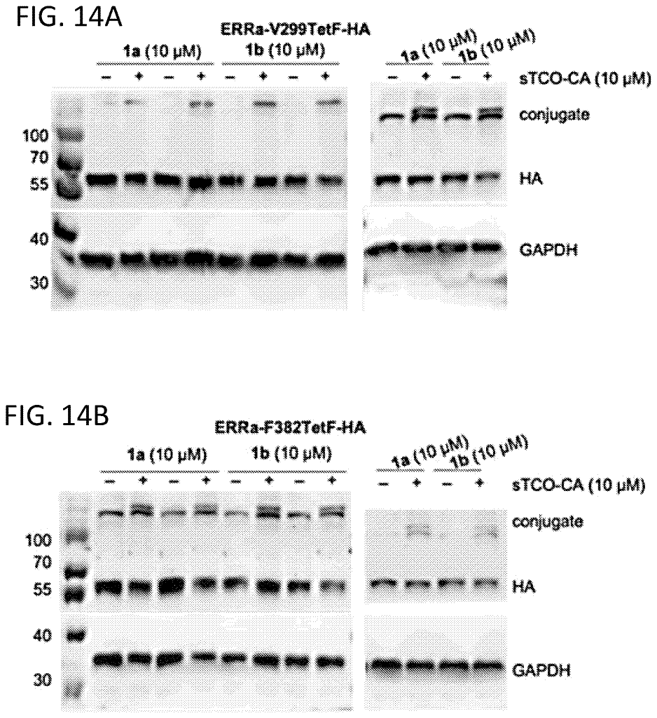

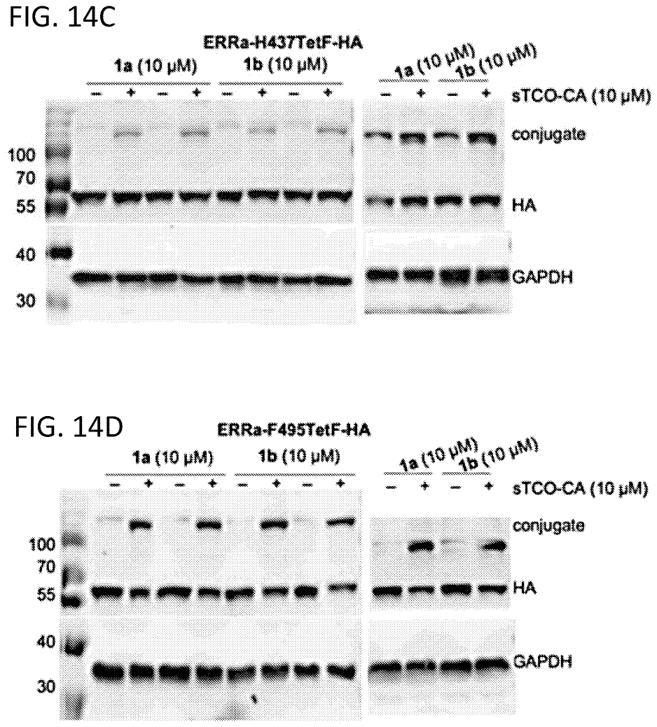

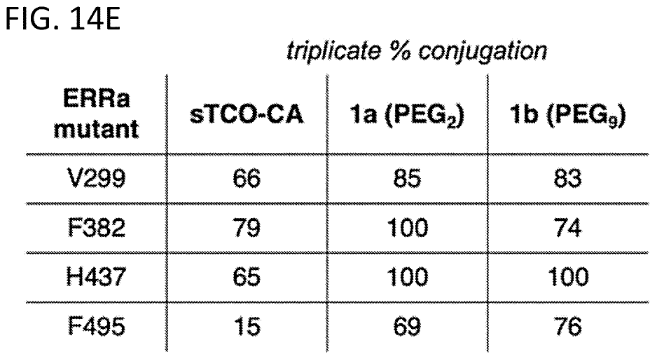

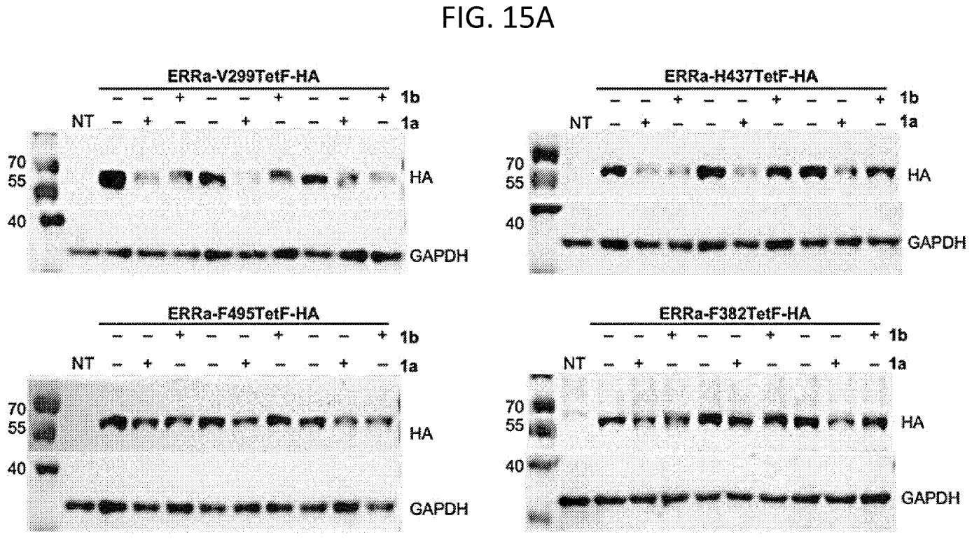

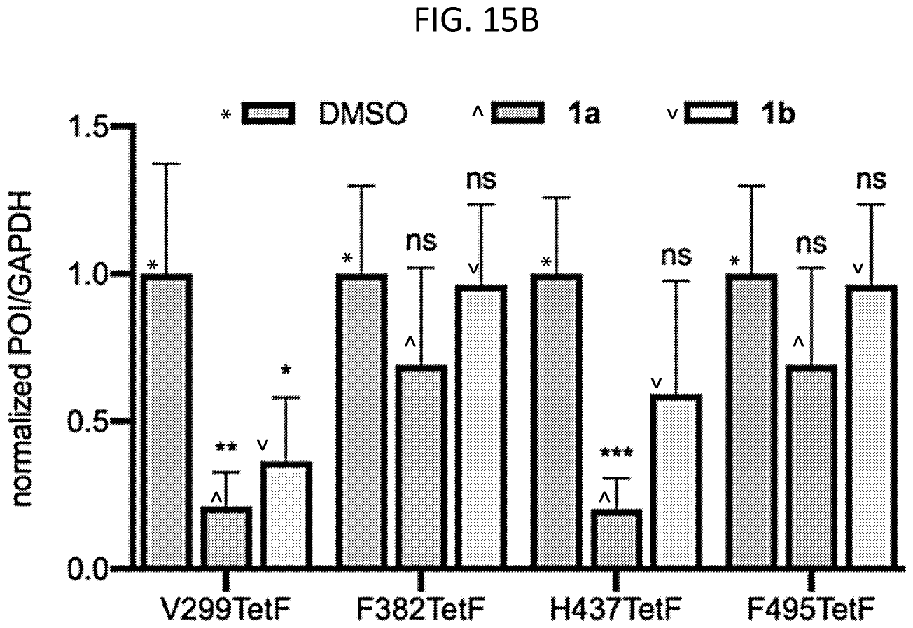

To test these hypotheses, four mutants of ERRα for the genetic incorporation of TetF on the protein surface were generated. Two mutants, F382TetF and F495TetF, were selected for their role in docking the known ERRα PROTAC (FIG. 6A) for direct comparison of PROTAC versus bioorthogonal E3 ligase ligand degradation. Two additional mutants, V299 and H437, were selected because of their proximity to surface lysine residues and their relative distance from the known PROTAC binding location. Bioconjugation efficiency of each mutant was quantified using sTCO-CA (FIG. 13), sTCO-PEG₂-CRBN (FIG. 14), and sTCO-PEG₂-CRBN (FIG. 14) before moving to degradation studies. Following treatment of each mutant with sTCO-PEGₙ-CRBN, interesting trends were observed. The two PROTAC binding pocket mutants, F382TetF and F495TetF demonstrated no significant degradation by either ligand (FIGS. 6B-D), despite demonstrating excellent bioconjugation (70-80%, FIG. 6D). The two surface residues not associated with the PROTAC binding region, V299TetF and H437TetF, were both significantly degraded by sTCO-PEG₂-CRBN, and V299TetF further demonstrated moderate degradation after treatment with sTCO-PEG₉-CRBN (FIGS. 6B-D). The observed differences in degradation for the four different mutants of ERRα indicate that more potent PROTACs could be designed that target different regions of the protein surface.

In addition to the ability to investigate specific regions of the protein surface for optimization of degradation efficiency, our bioorthogonal E3 ligase ligand can be used as an OFF switch to study protein function. The approach disclosed herein bypasses the need for protein-specific small molecules required for both classical and covalent PROTAC approaches, lessening the required synthetic workload and streamlining the study of novel and promiscuous proteins. Further, knock down proteins is achieved without the need for protein fusions to recruit heterobifunctional degraders, such as is required by systems like dTAGs, which require expression of FKBP alongside the protein target for inducible degradation, or the smaller peptide-based HiBiT-SpyTag system. The small, site-specific induction of degradation available through the methods disclosed herein allows for less perturbation of native protein functions for accurate and straightforward analysis of protein function.

### EXAMPLES

### 1. Biological Protocols

1-1a. Plasmid Construction: General Protocol

All site-directed mutagenesis reactions were performed using the same protocol with varying primers, template DNA, annealing temperatures, and elongation times. Mutagenesis was completed in a 50 μL reaction containing template DNA (10 ng), 10 μL of 5×NEB GC buffer (Thermo Scientific, F530L), 1 μL of dNTP mix (10 mM, Thermo Scientific, R0191), 1 μL of both forward and reverse primers (5 μM, Sigma Aldrich), 0.75 μL of DMSO, and 0.5 μL of Phusion polymerase (2 U/μL, Thermo Scientific, F530L). The samples were amplified using an initial denaturing step (95° C., 3 minutes), followed by 30 cycles of denaturing (95° C., 30 seconds), annealing (temperature varies, 3 minutes), and elongation (72° C., time varies) steps. The thermocycler program was completed with a final elongation step (72° C., 10 minutes), gradually cooled (16° C., 5 minutes), and the samples were stored at room temperature until further use. Template DNA was digested by adding 5 μL 10× CutSmart buffer and 1 μL of DpnI (20 U/μL, NEB, R0176S) to the 50 μL PCR mixture, and incubating for 1 hour at 37° C.

To assess the purity of the PCR product, 2 μL of the mixture was diluted in 8 μL of water and 2 μL of 6× loading dye (NEB, B7024S) and run on a 0.8% (w/v) agarose gel in Tris-buffered EDTA (TBE, 10 mM Tris-HCl, 1 mM EDTA, pH=8.0) buffer at 80 V for 30 minutes. If a single band corresponding to the correct length was observed, the mixture was used immediately for transformation. If impurities were observed, the PCR product was purified by gel extraction.

To gel extract, 10 μL of 6× loading dye was added to the DpnI-treated PCR mixture, and the entire volume was run on a 0.8% (w/v) agarose gel. The band corresponding to the correct length was cut from the gel and purified with the GeneJET Gel Extraction Kit (Thermo Scientific, K0692) according to the manufacturer's protocol.

To transform cells, a 50 μL aliquot of Top10 competent cells was thawed on ice in a 1.7-mL microcentrifuge tube (Laboratory Products Sales, L211511) and combined with 5 μL of the crude PCR mixture, or 75 ng of purified plasmid. The mixture was incubated on ice for 30 minutes. The cells were heat shocked by incubating the tube in a hot water bath (42° C., 30 s), followed immediately by incubation on ice for 2 minutes. Ice-cold SOC media (200 μL, VWR, 100219-988) was applied to the cell mixture, and the tube was incubated at 37° C. with shaking at 250 RPM for 1 hour. The entire contents of the transformation mixture were plated on 10 mL of LB agar supplemented with 10 μL of a 100 mg/mL antibiotic (ampicillin or kanamycin) stock and incubated overnight at 37° C. Three colonies were inoculated into 5 mL of LB broth supplemented with 5 μL of a 100 mg/mL antibiotic (ampicillin or kanamycin) stock and grown overnight with shaking (37° C., 250 rpm), followed by DNA miniprep (Thermo Scientific, K0503). Plasmid sequences were confirmed by Genewiz Sanger sequencing using universal CMV forward and EGFP-C reverse primers (P1 and P2, respectively, FIG. 6).

All Gibson assembly cloning procedures were preformed using the following protocol with varying template DNA, annealing temperatures, and elongation times. Backbone and insert amplifications were completed in a 100 μL reaction containing template DNA (20 ng), 20 μL of 5×NEB HF buffer, 2 μL of dNTP mix (10 mM, each, Thermo Scientific, R0191), 1 μL each of forward and reverse primers (10 UM, each, Sigma Aldrich), 1.5 μL of DMSO, and 1 μL of Phusion polymerase (2 U/μL, Thermo Scientific, F530L). The thermocycler program outlined above was used for insert and backbone amplification, and the products were gel extracted as described above. The DNA fragments were annealed using the published Gibson assembly method.¹ The 5× isothermal reaction buffer was prepared (25% PEG-8000 (Alfa Aesar), 500 mM Tris-HCl (amresco) pH 7.5, 50 mM MgCl₂ (Alfa Aesar), 50 mM DTT (Fisher), 1 mM each of the 4 dNTPs (Fisher), and 5 mM NAD (NEB)) and stored at −20° C. The master mix was prepared (86 μL of autoclaved MilliQ water, 36 μL of 5× isothermal reaction buffer, 2 μL of Phusion DNA polymerase (2 U/μL Fisher), 0.064 μL of T5 exonuclease (10 U/μL, NEB), and 0.016 μL of Taq ligase (40 U/μL, abm)) and stored at −20° C. in 15 μL aliquots before use. Final DNA products were transformed, purified, and sequenced as described above.

1-1b. Plasmid Construction: pmCherry-EGFP-TAG

Using the Gibson assembly protocol outlined in the Plasmid Construction: General Protocol section, the mCherry gene was amplified from pmCherry-N1 (Clontech, 632523) using primers P3 and P4 (annealing temp=59.9° C., elongation time=30 seconds) and cloned into the pEGFP-HA backbone (amplified with P5 and P6, annealing temp=60° C., elongation time=3 minutes) for assembly of pmCherry-EGFP-HA. The site-directed mutagenesis protocol described in section 1.1a was used to introduce the following mutations:
Y151TAG (P7 and P8, annealing temp=56.1° C., elongation time=3.5 minutes)
I128TAG (P9 and P10, annealing temp=65.5° C., elongation time=3.5 minutes)
G134TAG (P11 and P12, annealing temp=63.2° C., elongation time=3.5 minutes)
G191TAG (P13 and P14, annealing temp=67° C., elongation time=3.5 min)
A206TAG (P15 and P16, annealing temp=64° C., elongation time=3.5 min)
   1-1c. Plasmid Construction: pMKP3-HA-TAG

EGFP was cloned out of pWT-MKP3-EGFP-HA using primers P17 and P18 (annealing temp=62.6° C., elongation time=3 minutes) to yield pWT-MKP3-HA. The site-directed mutagenesis protocol described in 1-1a was used to introduce the following mutations into pcaMEK1-EGFP-HA:
L246TAG (P19 and P20, annealing temp=60.1° C., elongation time=3 minutes)
S265TAG (P21 and P22, annealing temp=61.1° C., elongation time=3 minutes)
S277TAG (P23 and P24, annealing temp=61.0° C., elongation time=3 minutes)
G338TAG (P25 and p26, annealing temp=60.2° C., elongation time=3 minutes)
   1-1d. Plasmid Construction: pERRα-HA-TAG

Using the Gibson assembly protocol outlined in the Plasmid Construction: General Protocol section, the ERRα gene was amplified from pCMV-flag-ERRalpha (Addgene, 10975) using primers P27 and P28 (annealing temp=61.1° C., elongation time=1.5 minutes) and cloned into the pEGFP-HA backbone (P29 and P30, annealing temp=63.5° C., elongation time=3 minutes) for assembly of pERRα-EGFP-HA. The EGFP gene was removed through using primers P31 and P32 (annealing temp=61° C., elongation time=3 minutes) for generation of pERRα-HA. The site-directed mutagenesis protocol described in section 1.1a was used to introduce the following mutations:
V299TAG (P33 and P34, annealing temp=66.2° C., elongation time=3 minutes)
F382TAG (P35 and P36, annealing temp=66° C., elongation time=3 minutes)
H437TAG (P37 and P38, annealing temp=66° C., elongation time=3 minutes)
F495TAG (P39 and P40, annealing temp=62.2° C., elongation time=3 minutes)

### 1-2. Cell Culture

All cell culture experiments were performed in a sterile laminar flow hood. HEK293T cells (ATCC, CRL-11268) were maintained in Dulbecco's Modified Eagle Medium (DMEM, Gibco, SH30003.03) supplemented with 10% (v/v) fetal bovine serum (Sigma Aldrich, F0926) and 1% (v/v) penicillin/streptomycin (complete medium, Corning, 30002CI) at 37° C. with 5% CO₂. Cells were used between passage number 3 and 32 and tested for Mycoplasma contamination every 6 months (Genlantis, MY01100). All cell culture medias are warmed to 37° C. prior to application to cells.

### 1-3. Western Blot

HEK293T cells were seeded into a polyD-lysine (PDK) treated 48-well plate (50,000 cells/well, Greiner, 665180) in 250 μL of complete medium. When cells reached 80-90% confluency, the media in each well was replaced with 250 μL of fresh, antibiotic-free DMEM. The media was supplemented with 0.25 mM tetrazine phenylalanine (TetF2) by diluting 12.5 μL of a 100 mM stock solution into 5 mL of antibiotic-free DMEM to create a master mix solution containing a final concentration of 0.25% DMSO. The media in separate wells was replaced with 200 μL of the UAA-containing media.

A master mix transfection solution was prepared by combining 400 ng of DNA per well (3:1 TAG-mutant/synthetase in 40 μL of OptiMEM transfection media (Thermo Scientific, 22600050) and 2 μL of 1 mg/mL linear polyethyleneimine (LPEI, Polysciences, 23966) per well. For example, a six well transfection master mix contained 800 ng of TAG-containing plasmid (pmCherry-EGFP-TAG-HA, pMKP3-HA-TAG, or pERRα-HA-TAG) and 1600 ng of pAcBac-TetFRS diluted into 240 μL of OptiMEM and 12 μL of LPEI. The transfection master mix was gently mixed by inversion and incubated for 10 minutes at room temperature. Following incubation, 40 μL of the mixture was added dropwise to each well. The cells were then incubated at 37° C. and 5% CO₂. for 24 hours.

Following overnight transfection, the cells were washed by gently applying 200 μL of fresh complete medium to each well and immediately removing the wash. A second wash was completed by adding another 200 μL of fresh complete medium to each well and incubating the cells at 37° C. for 30 minutes.

For sTCO-PEGₙ-CRBN dose-response experiments (FIG. 3A), a 250 μM working solution of sTCO-PEGₙ-CRBN was made by diluting 25 μL of a 10 mM stock (in DMSO) into 975 μL of complete medium. To make a 100 μM mixture, 200 μL of the 250 μM stock was diluted into 300 μL of complete medium. Further 10× serial dilutions were made by diluting 50 μL of the 100 μM solution into 450 μL of complete medium. This serial dilution was repeated to generate the designated concentrations. Following the wash steps above, 200 μL of media supplemented with or without sTCO-PEGₙ-CRBN was applied to each well and the plate was incubated at 37° C. 5% CO₂. overnight.

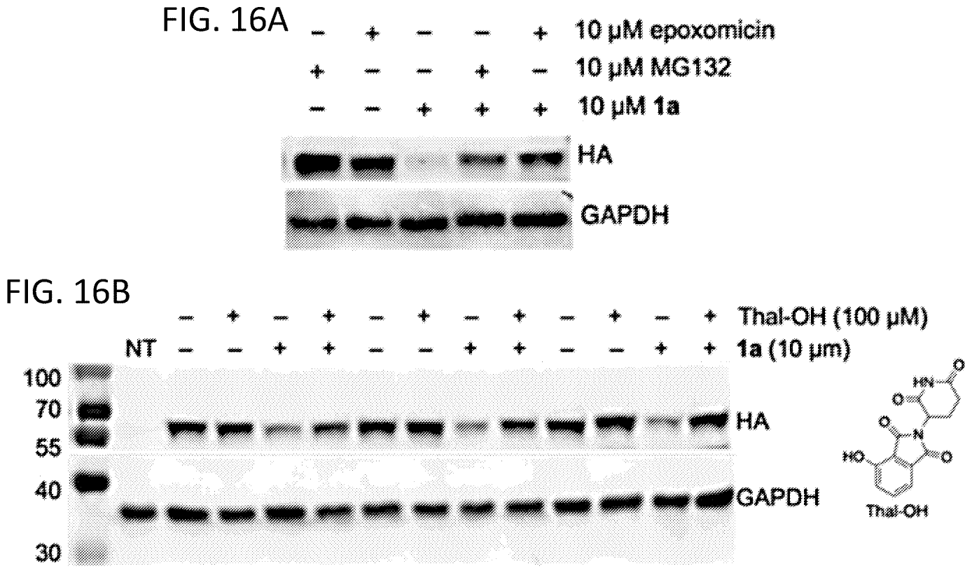

For mechanistic insight into sTCO-PEG₂-CRBN degradation (FIG. 16) cells were transfected as described above. A 10 μM working solution of media containing either MG132 or epoxomicin was made by adding 1 μL of the respective proteasome inhibitor (10 mM in DMSO) into 999 μL of complete medium. Following removal of TetF, 200 μL of media supplemented with either MG132, epoxomicin, or DMSO was added to each well and the cells were incubated for 4 h. Next, a solution containing 50 μM of sTCO-PEG₂-CRBN was made by adding 5 μL of sTCO-PEG₂-CRBN (10 mM in DMSO) into 995 μL of complete medium. Following the allotted time for proteasome inhibitor incubation, 50 μL of 50 μM sTCO-PEG₂-CRBN was added directly to each well, for a final volume of 250 μL and final concentration of 10 μM of sTCO-PEG₂-CRBN. The cells were then incubated overnight at 37° C. with 5% CO₂ before lysis and western blot analysis.

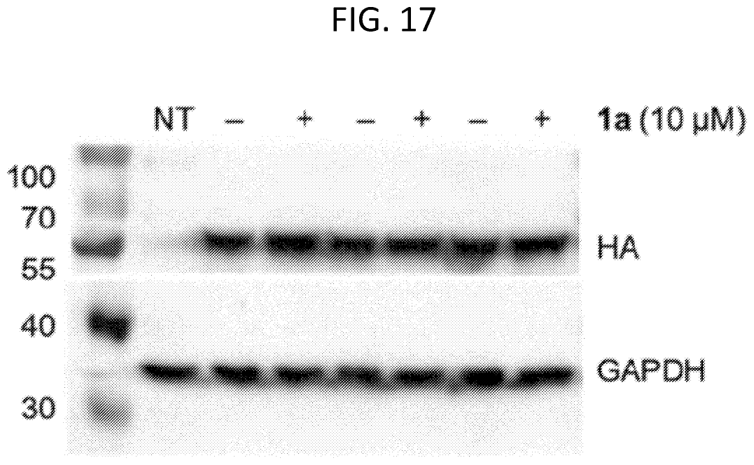

Tetrazine specificity of sTCO-PEG₂-CRBN (FIG. 17) was confirmed following transfection of HEK293T cells with wild-type pmCherry-EGFP-HA (no TAG mutant). Transfection was performed similarly to the method described above. Instead, no TetF was included in the antibiotic-free DMEM, only 133 ng of pmCherry-EGFP-HA was added per well, and no additional synthetase plasmid was included in the transfection mastermix. Following overnight expression, cells were similarly washed before incubation with 10 μM of sTCO-PEG₂-CRBN overnight. Cells were lysed and resultant protein levels were analyzed by western blot.

For sTCO-PEG₂-CRBN hook effect mitigation (FIGS. 3B-C), the same concentrations of sTCO-PEG₂-CRBN were made using the same dilutions above. For each condition, 200 μL of supplemented media was added to the respective well and incubated for either 15 or 60 minutes. Following the specified incubation, the media containing sTCO-PEG₂-CRBN was removed, and 200 μL of fresh complete medium was applied. The cells were then incubated overnight at 37° C. with 5% CO₂.

Following the indicated incubation times, each well was washed with 120 μL of cold PBS to remove residual medium. The plate was placed on ice and to each well was added 60 μL of cold RIPA buffer (50 mM Tris-HCl, pH 8.0, 150 mM NaCl, 1% (v/v) NP-40, 0.5% (w/v) sodium deoxycholate, 0.1% (w/v) SDS) supplemented with 0.6 μL of 100× protease inhibitor cocktail (Thermo Scientific, 78429) with 250 RPM shaking for 15 minutes. Cell debris was removed from the plate and the cell lysate (60 μL) was combined with 20 μL of 4×SDS-PAGE sample loading buffer (200 mM Tris-HCl, pH 6.5, 400 mM DTT, 8% (w/v) SDS, 6 mM bromophenol blue, 4 M glycerol). The samples were heated at 95° C. for 10 minutes, separated by 10% (v/v) SDS-PAGE gel electrophoresis (80 V for 20 minutes, 150 V for 80 minutes) with an ice pack placed inside the tank next to the gel cassette. Completed gels were transferred to a 0.45 μm PDVF membrane (Millipore, IPVH00010) at 80 V for 2 hours using cold 1× Transfer Buffer (25 mM Tris-HCl, 192 mM glycine, pH=8.3, 20% methanol), in an ice bath.

The membrane was blocked for 1 hour with 3 mL of blocking buffer (5% milk in TBS with 0.1% (v/v) Tween 20, TBST) at room temperature with rocking. Blots were probed with rabbit mAb anti-HA (1:1000 dilution, CST, 3724S) and rabbit pAb anti-GAPDH (1:10000 dilution, ProteinTech, 50-172-6351) primary antibodies in 3 mL of blocking buffer overnight with rocking at 4° C. After washing three times by incubating the blots with 10 mL of cold TBST with rocking, membranes were incubated for 1 hour with goat anti-rabbit IgG HRP-linked secondary antibody (1:5000 dilution, CST 7074S) in 3 mL of TBST. After washing three times with 3 mL of cold TBST with rocking, the blots were developed in either SuperSignal West Pico Chemiluminescent Substrate (Thermo Scientific, 34580) or homemade imaging substrates (Solution A: 39 mg of luminol and 47.5 mg of p-iodophenol in 50 mL of 0.1M Tris-HCl, pH 9.35, and Solution B: 5 μL of 30% hydrogen peroxide in 50 mL of 0.1M Tris-HCl, pH 9.35). For either chemiluminescent substrate condition, 2 mL of the luminol/enhancer solution was mixed with 2 mL of the peroxide solution and incubating the membrane in the resulting solution at room temperature with rocking for 5 minutes. The blots were imaged on a BioRad ChemiDoc system with automated exposure times. Western blots were quantified using ImageJ software. Lanes were assigned and the protein of interest (POI) and GAPDH bands were identified and quantified relative to the minus compound control. Standard error was determined from three biological replicates.

The blots were imaged on a BioRad ChemiDoc system with automated exposure times. Western blots were quantified using ImageJ software: The first individual band/lane was selected using the Rectangular Selection tool, then denoted as “First Lane” through Analyze>Gels>Select First Lane. The newly generated box was positioned over the subsequent lane and denoted as the “Next Lane” through Analyze>Gels>Select Next Lane, and repeated until all bands/lanes were accounted for. Each lane was plotted using Analyze>Gels>Plot Lanes. On the generated graphs, a baseline was added using the Straight Line tool, then the area under the curve was quantified using the Wand tool. The values obtained for each POI were transferred to Excel, then normalized to their respective loading controls (GAPDH), calculated the same way. Then, each POI/GAPDH value was normalized to the average of each respective minus compound controls. Standard error was determined from three biological replicates using one- or two-way ANOVA via Prism 9.

For the pulse chase experiment, the initial treatment with the compound of interest (e.g., 1) will react with the protein of interest. The subsequent treatment with sTCO-CA will lead to reaction with any remaining protein not already conjugated by 1. Cell lysis and subsequent incubation with HaloTag will then form a heterodimer with any protein conjugated by sTCO-CA, and no dimer will form if all protein-tetrazine has been bioconjugated by sTCO-PEGₙ-CRBN. Western blot analysis shows a shift in molecular weight when the HaloTag heterodimer is formed. Therefore, the amount of bioconjugation to sTCO-PEGₙ-CRBN is represented by the amount of non-HaloTag modified protein, meaning the band with the same molecular weight as the negative control. As this experiment is done in triplicate, the average amount of non-conjugated protein for each pulse chase experiment is divided by the average amount of the negative control, and thus represents the percent of bioconjugation.

### 1-4. Docking

The small molecule ligand BCI was docked into DUSP6 (PDB 1MKP) according to literature protocols^{[8]} using Chimera and Autodock Vina. The protein structure of DUSP6 was retrieved from the RCSB protein database using its PDB code. The structure was prepared for docking using Tools>Structure Editing>Dock Prep. In the new window, the following options were selected: Delete solvent, If alternate locations, keep only highest occupancy, Incomplete side chains: Replace using Dunbrack rotamer library, Add hydrogens, Add charges, and Write Mol2 file. In the next “Add Hydrogens” window, no parameters were changed. In the next, “Assign Charges for Dock Prep” window, Gasteiger charges were selected. No changes were made in the following window, “Specify Net Charges.” The prepared structure was saved in a separate file. The BCI ligand was then prepared in Chimera under Tools>Structure Editing>Build Structure. The SMILES code (O=C(/C(C1NC2CCCCC2)=C/C3=CC=CC=C3)C4=C1C=CC=C4) was added and applied to the structure. To then dock BCI into the region outlined in the literature, Tools>Surface/Binding Analysis>Autodock Vina. Under “Receptor search volume options” the box parameters were set to Center: 6, 54, 5 and Size: 12, 13, 10. No other parameters in Autodock Vina were changed.

### 2. Synthetic Protocols

### 2-1. General Chemical Methods

All reagents and solvents were purchased from commercial suppliers and used without further purification. All reactions were performed in flame-dried glassware and stirred magnetically. Flash chromatography was performed by hand. NMR spectra were recorded on Bruker Ultrashield 300 MHz, 400 MHz, or 500 MHz spectrometers. Analytical LC-MS data were collected on a Shimadzu LCMS-2020 and a Thermoscientific Q-Exactive Orbitrap. High resolution mass spectrometry was performed by the University of Pittsburgh facilities.

### Abbreviations Used

DCM=dichloromethane
DMF=dimethylformamide
TLC=thin layer chromatography
sTCO=strained trans-cyclooctene
EtOAc=ethyl acetate
HATU=Hexafluorophosphate Azabenzotriazole Tetramethyl Uronium
DIPEA=diisopropylethylamine
TFA=trifluoroacetic acid
MeOH=methanol
   2-2. Synthesis of sTCO-PEGₙ-CRBN

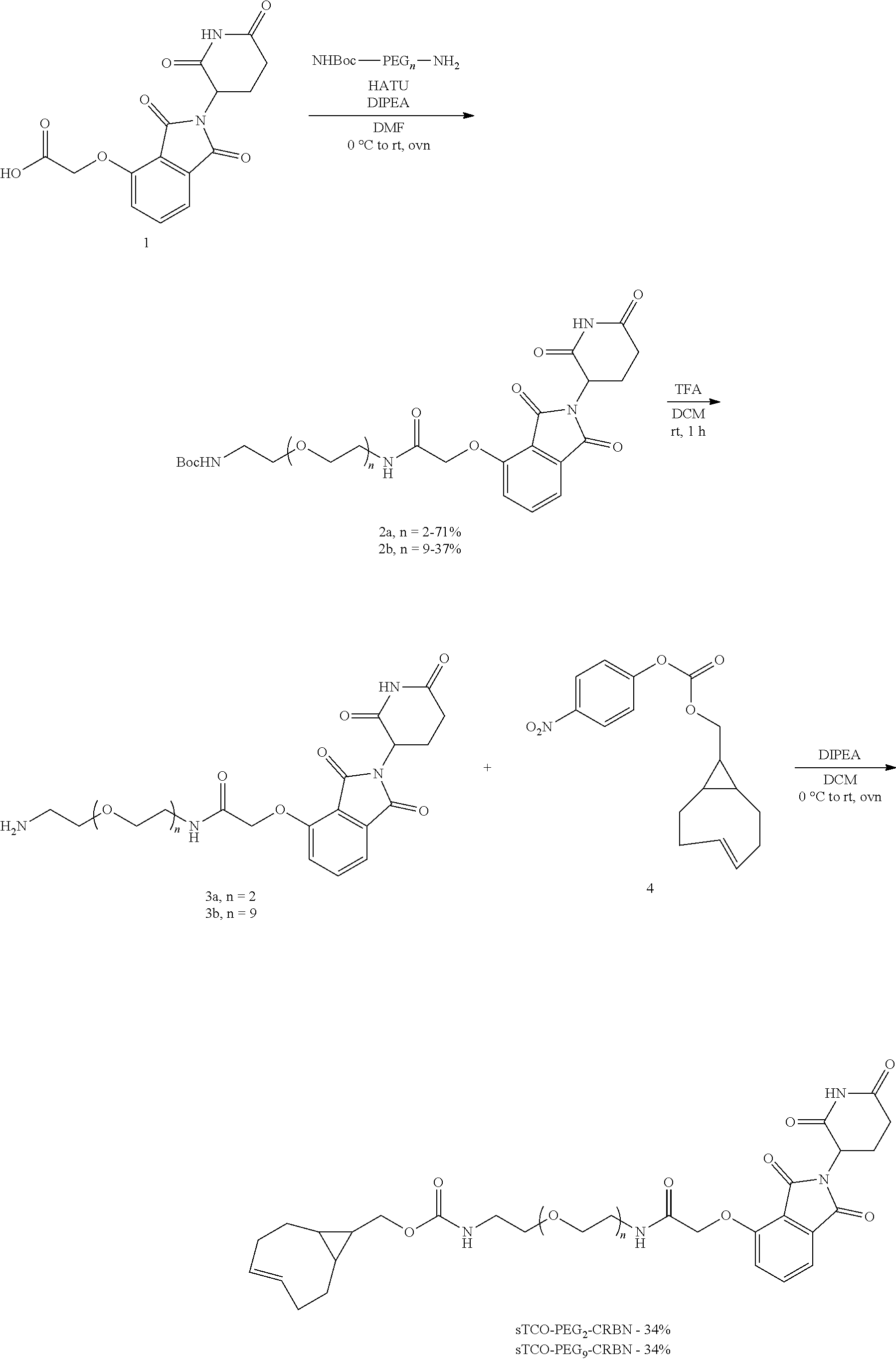

### General Procedure

Compound 1 was synthesized and characterized according to the literature. The carboxylic acid (1 eq) was dissolved in DMF (final carboxylic acid concentration of 0.1 M), then HATU (1.1 eq) and DIPEA (4 eq) were added, and the mixture was stirred on ice for 5 minutes. The NHBoc-PEGₙ-Amine (1.1 eq) was added last, and the mixture was allowed to warm to room temperature overnight. Following consumption of the carboxylic acid as observed on TLC (5% MeOH/DCM), the reaction was diluted with brine (10 times the volume of DMF used in the reaction) and extracted four times with EtOAc (5 mL). The combined organic layers were dried over sodium sulfate and concentrated in vacuo. The resultant crude oil was subjected to normal phase flash chromatography, using 5% MeOH/DCM to elute the product (2).

Following characterization of the PEGylated product, the Boc-protected material was dissolved in 25% TFA in DCM and stirred at room temperature open to the air. When complete consumption of the Boc-protected starting material was observed by TLC (usually 30-60 minutes), the reaction was concentrated in vacuo. Excess TFA was removed by resuspending the crude material in methanol and concentrating to dryness, then repeating this process 3 times. The deprotection was characterized by LC-MS, then 3 was used directly in the next step.

Compound 4 was synthesized and characterized according to the literature, then added as a neat solid (1.2 eq) to a solution of 3 in DCM (final reaction concentration of 0.01 M) and DIPEA (4 eq). The reaction was kept under foil and stirred at room temperature overnight. Following consumption of the amine starting material, the reaction was concentrated in vacuo. The crude material was subjected to normal phase flash chromatography (5 to 10% MeOH/DCM). The resultant material collected contained 4-nitrophenol impurities, which were not separable by flash chromatography. To remove this impurity, the mixture was dissolved in 10 mL of DCM and extracted with small amounts of saturated sodium carbonate (2-5 mL). The resultant, pure product was characterized by 1H NMR, 13C NMR, LC-MS and HRMS.

NH₂-PEG₂-CRBN (2a)—¹H NMR (400 MHz, CDCl₃) δ

NH₂-PEG₉-CRBN (2b)—¹H NMR (400 MHz, CDCl₃) δ 8.89 (bs, 1H), 7.75 (t, 1H, J=7.6 Hz), 7.68 (m, 1H), 7.54 (d, 1H, J=4.8 Hz), 7.22 (d, 1H, J=8.4 Hz), 6.14 (bs, 1H), 4.95 (m, 1H), 4.66 (s, 2H), 3.60 (m, 32H), 3.52 (m, 4H), 3.38 (m, 2H), 3.31 (m, 4H), 3.18 (m, 2H), 3.0 (bs), 2.16 (m, 1H), 1.5 (s, 9H).

sTCO-PEG₂-CRBN—¹H NMR (500 MHz, CDCl₃) δ 8.68 (bs, 1H), 7.73 (t, 1H, J=7.5 Hz), 7.61 (m, 1H), 7.55 (d, 1H, J=7 Hz), 7.19 (d, 1H, J=8.5 Hz), 6.85 (m, 1H), 5.35 (m, 1H), 5.11 (1H, m), 4.96 (dd, 1H, J=5.5, 5), 4.66 (s, 2H), 3.93 (d, 2H, J=6 Hz), 3.60 (m, 11H), 3.34 (s, 2H), 2.83 (m, 3H), 2.14 (m, 8H), 1.42 (m, 1H), 1.26 (m, 5H), 0.85 (m, 2H), 0.52 (m, 2H), 0.40 (m, 2H). ¹³C NMR (500 MHz, CDCl₃) 171.2, 168.3, 167.1, 166.8, 166.0, 157.2, 154.6, 138.5, 137.1, 133.8, 131.4, 126.3, 119.5, 118.2, 117.5, 115.8, 70.4, 70.3, 70.2, 69.7, 68.1, 53.6, 49.4, 40.9, 39.2, 38.8, 33.9, 32.7, 31.5, 29.8, 29.7, 29.5, 29.4, 27.8, 24.8, 22.8, 22.1, 21.1, 14.3. HRMS (M+H)⁺ calcd for C₃₂H₄₀N₄O₁₀ (M+H)⁺ 640.2744. found 639.2639.

sTCO-PEG₉-CRBN—¹H NMR (500 MHz, CDCl₃) δ 8.95 (bs, 1H), 7.75 (t, 1H, J=7.5 Hz), 7.65 (m, 1H), 7.55 (d, 1H, J=7 Hz), 5.86 (m, 1H), 5.11 (m, 1H), 4.65 (s, 1H), 3.93 (s, 2H), 3.65 (m, 32H), 3.57 (m, 4H), 3.35 (s, 2H), 2.78 (m, 4H), 2.25 (m, 6H), 1.91 (m, 6H), 0.85 (m, 2H), 0.54 (m, 2H), 0.42 (m, 2H). ¹³C NMR (500 MHz, CDCl₃). HRMS (M+H)+ calcd for C₄₆H₆₈N₄O₁₇ (M+H)+948.4579. found 947.4507.

In view of the many possible embodiments to which the principles of the disclosed invention may be applied, it should be recognized that the illustrated embodiments are only preferred examples of the invention and should not be taken as limiting the scope of the invention.

1. A compound, or a stereoisomer, isotopomer, tautomer, or salt thereof having a structure of:
A-L-B
wherein A is a ligand that can covalently bioconjugate to a tetrazine-modified unnatural amino acid, or to a dienophile-modified unnatural amino acid, or to an azide-modified unnatural amino acid; L is a linker covalently attaching A and B; and B is a ligand that can bind to an E3 ligase.

2. A compound, or a stereoisomer, isotopomer, tautomer, or salt thereof having a structure of:
A-L-B
wherein A is a ligand that can covalently bioconjugate to a tetrazine-modified unnatural amino acid present on a protein, or to a dienophile-modified unnatural amino acid present on a protein, or to an azide-modified unnatural amino acid present on a protein; L is a linker covalently attaching A and B; and B is a ligand that can bind to an E3 ligase.

3. The compound of claim 1, wherein A includes a strained alkene or a strained alkyne.

4. The compound of claim 1, wherein A is selected from: 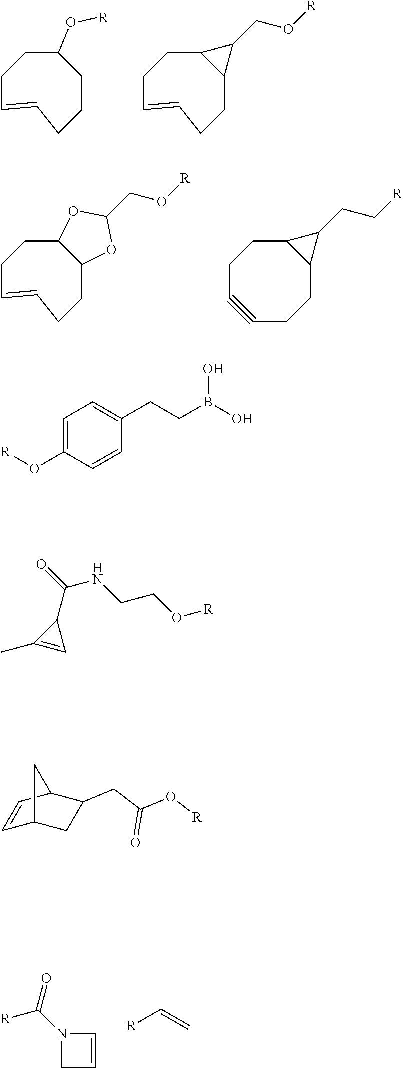
wherein R is attachment to L.

5. The compound of claim 1, wherein A is selected from: 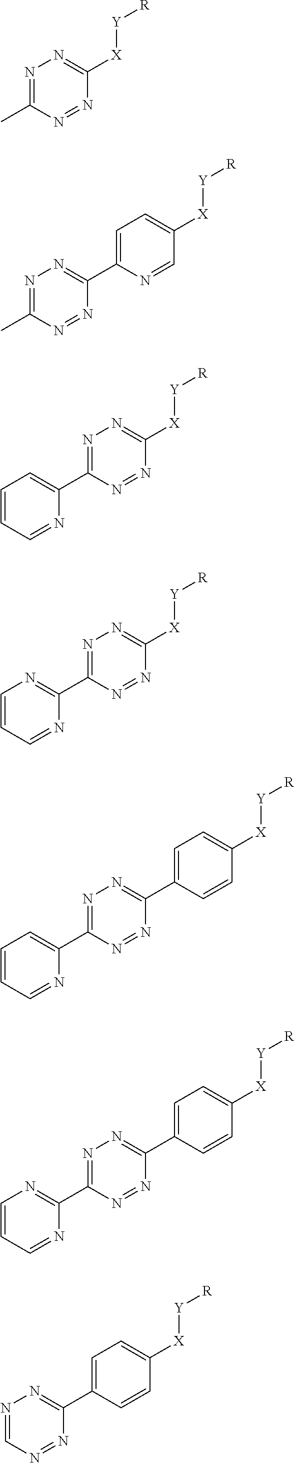
wherein X is CH₂ or C═O; Y is NH; and R is attachment to L.

6. The compound of claim 1, wherein A is selected from: 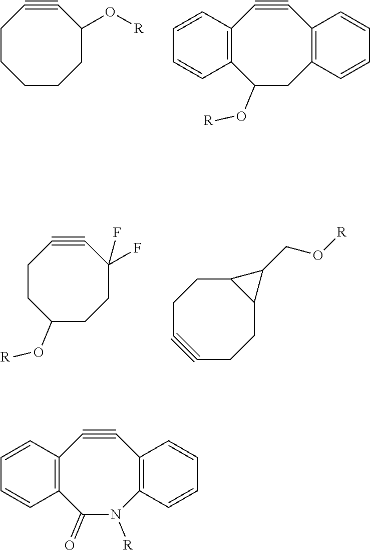
wherein R is attachment to L

7. The compound of claim 1 wherein L includes at least one of a glycol, an alkyl, an alkyne, an azobenzene, a triazole, a piperazine, or a piperidine.

8. The compound of claim 1, wherein B is a ligand that can bind to a E3 ligase for cereblon, VHL, MDM2, or IAP.

9. The compound of claim 1, wherein the compound has a structure of: 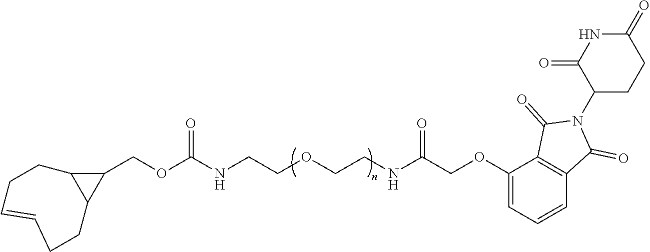
wherein n is 2 to 23.

10. The compound of claim 1, wherein B is a ligand that can bind to a E3 ligase for cereblon and the ligand is selected from:

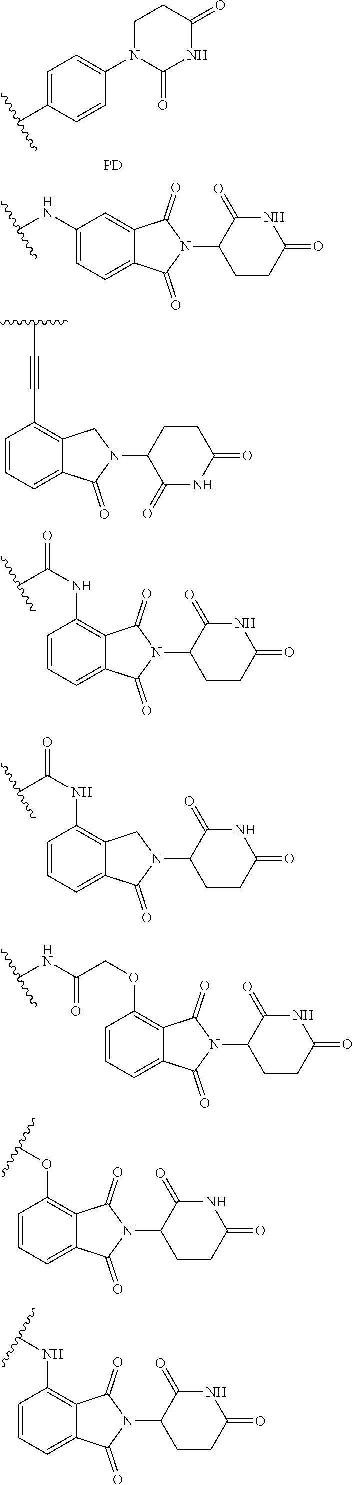

11. The compound of claim 1, wherein B is a ligand that can bind to a E3 ligase for VHL and the ligand is selected from:

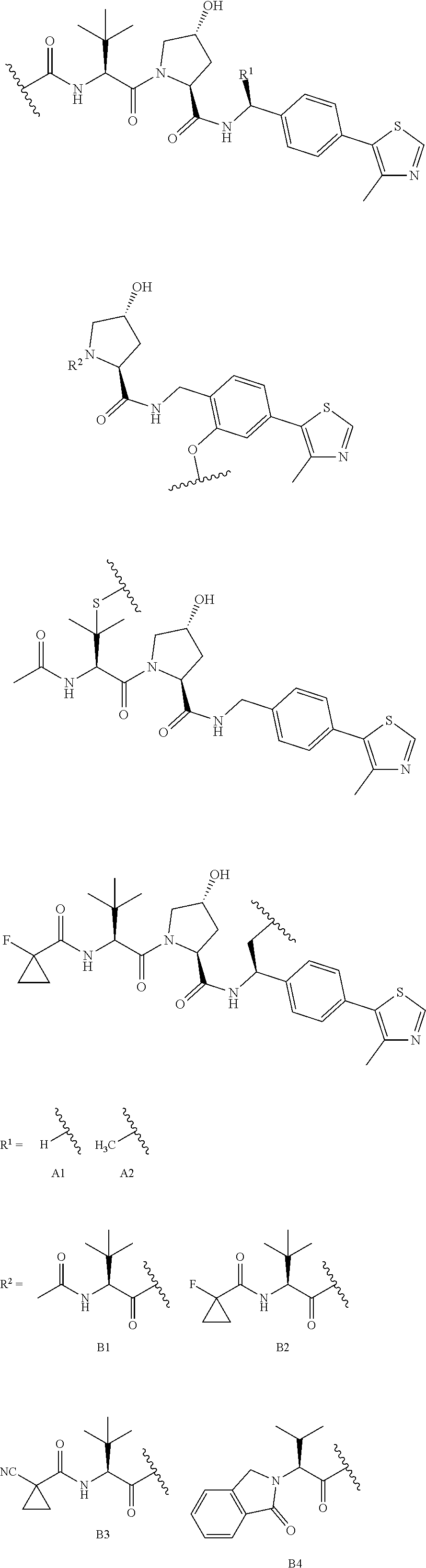

12. The compound of claim 1, wherein B is a ligand that can bind to a E3 ligase for MDM2 and the ligand is selected from:

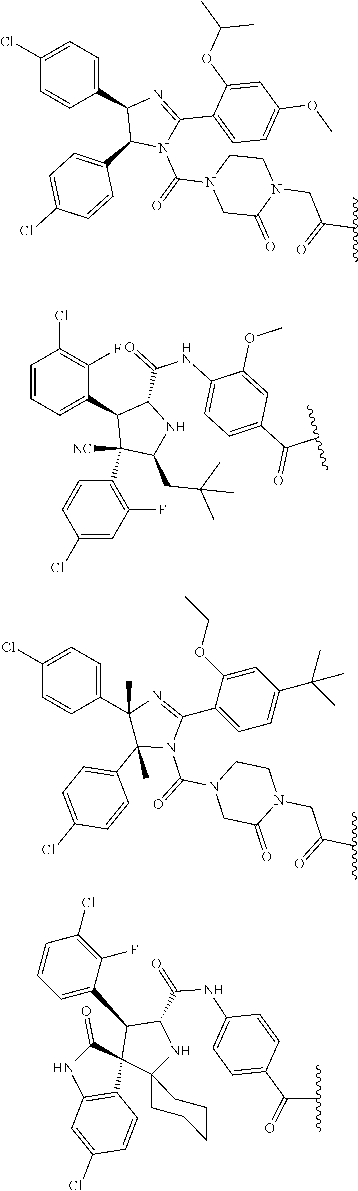 [SMILES: *C(=O)CN1CCN(C(=O)N2C(c3ccc(OC)cc3OC(C)C)=N[C@H](c3ccc(Cl)cc3)[C@@H]2c2ccc(Cl)cc2)CC1=O ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1=O ; *C(=O)c1ccc(NC(=O)[C@@H]2NC3(CCCCC3)[C@@]3(C(=O)Nc4cc(Cl)ccc43)[C@H]2c2cccc(Cl)c2F)cc1]

13. The compound of claim 1, wherein B is a ligand that can bind to a E3 ligase for IAP and the ligand is selected from:

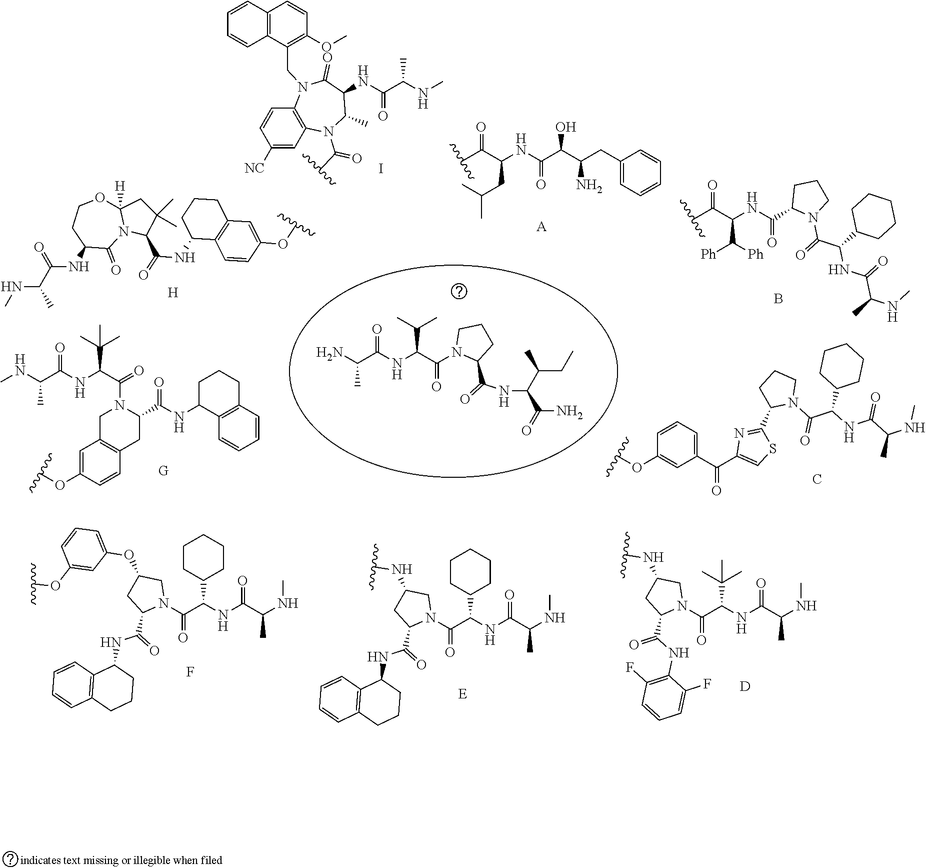

14. The compound of claim 1, wherein B is selected from: 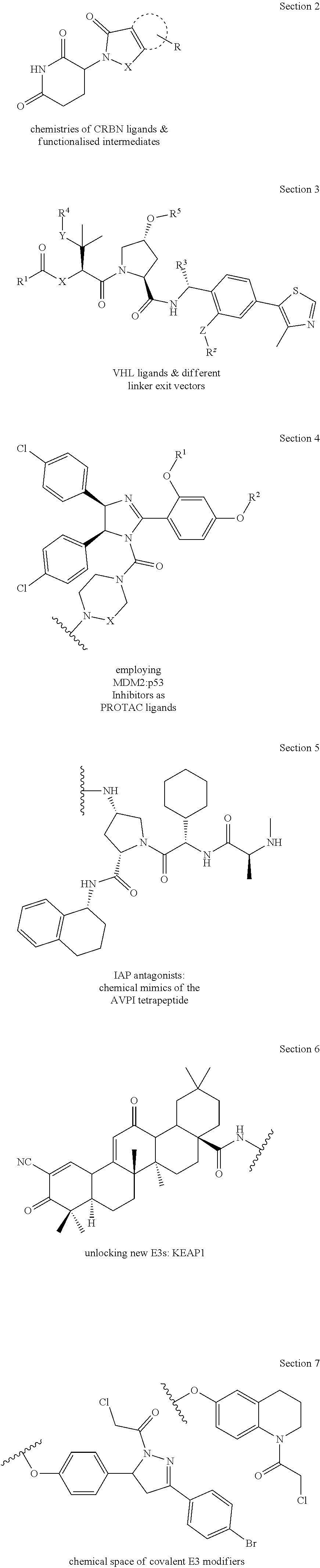
wherein for the CRBN ligand R is thiol, alcohol, alkyne, alkene, benzyl, amino, or amido; and X is H or C═O
wherein for the VHL ligand X is NH, Y is SR, wherein R is H or alkyl; Z is OH; R¹ is NHR, OR, or linker to L, wherein R is H or alkyl; R² is alternative linker to L; R³ is H or CH₃; R⁴ is alternative linker to L; and R⁵ is H or a caging group
wherein for the MDM2 ligand X is C═O or CH₂; R¹ is CH(CH₃)₂ or CH₂CH₃; and R² is CH₃ or an amide linker to L.

15. A method comprising:
covalently bioconjugating the compound of claim 1 to a protein of interest wherein the protein of interest includes a site-specific installed conjugation handle for the compound.

16. The method of claim 15, wherein the protein of interest is in a cell or organism that encodes an unnatural amino acid that includes the conjugation handle.

17. The method of claim 15, wherein the bioconjugation handle comprises a tetrazine.

18. The method of claim 15, wherein the unnatural amino acid that includes the conjugation handle has a structure selected from:

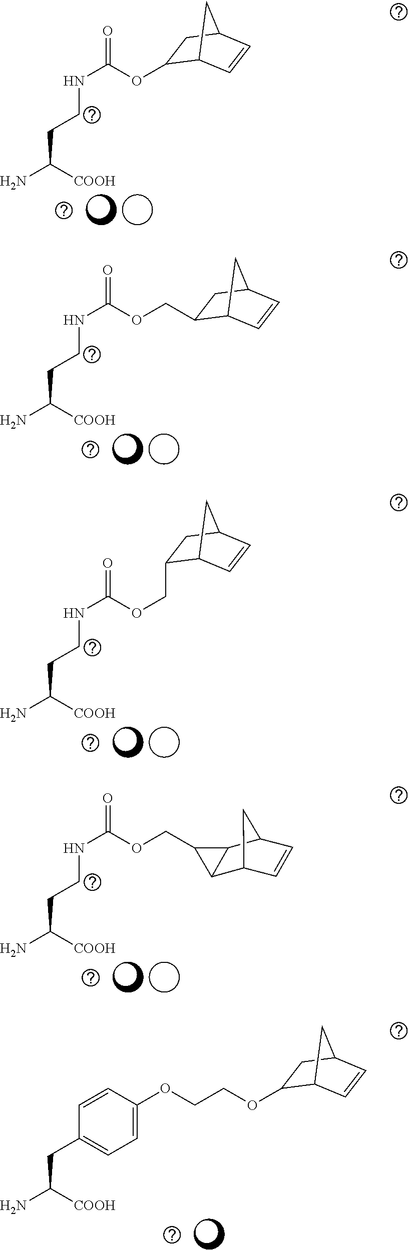 [SMILES: N[C@@H](CCNC(=O)OC1CC2C=CC1C2)C(=O)O ; N[C@@H](CCNC(=O)OCC1CC2C=CC1C2)C(=O)O ; N[C@@H](CCNC(=O)OCC1CC2C=CC1C2)C(=O)O ; N[C@@H](CCNC(=O)OCC1C2C3C=CC(C3)C12)C(=O)O ; N[C@@H](Cc1ccc(OCCOC2CC3C=CC2C3)cc1)C(=O)O]

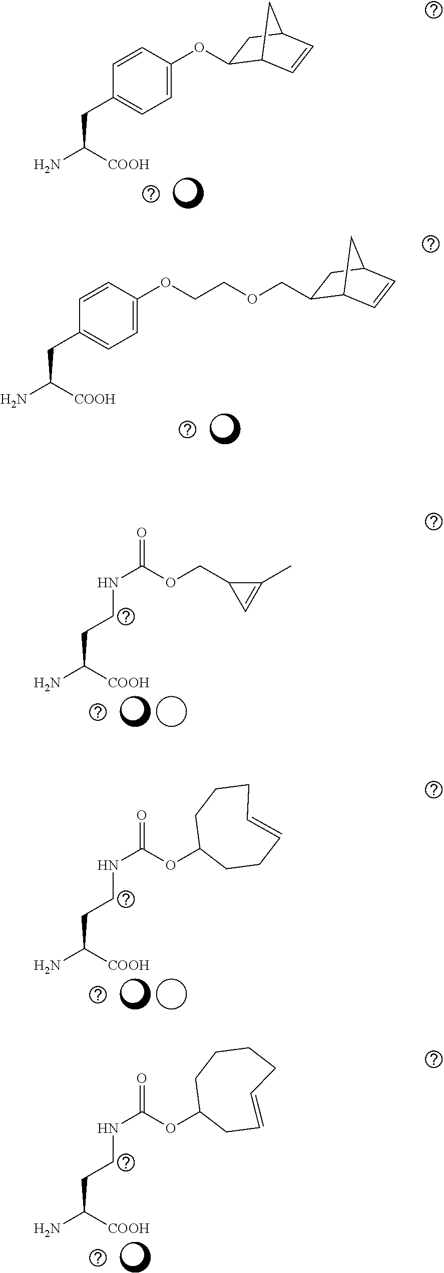 [SMILES: N[C@@H](Cc1ccc(OC2CC3C=CC2C3)cc1)C(=O)O ; N[C@@H](Cc1ccc(OCCOCC2CC3C=CC2C3)cc1)C(=O)O ; CC1=CC1COC(=O)NCC[C@H](N)C(=O)O ; N[C@@H](CCNC(=O)OC1CC/C=C/CCC1)C(=O)O ; N[C@@H](CCNC(=O)OC1C/C=C/CCCC1)C(=O)O]

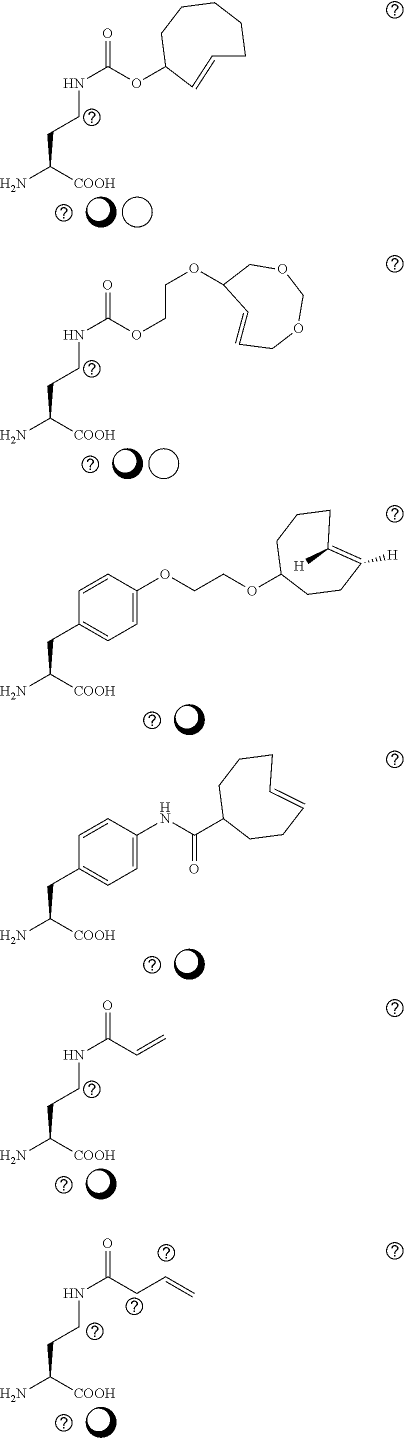

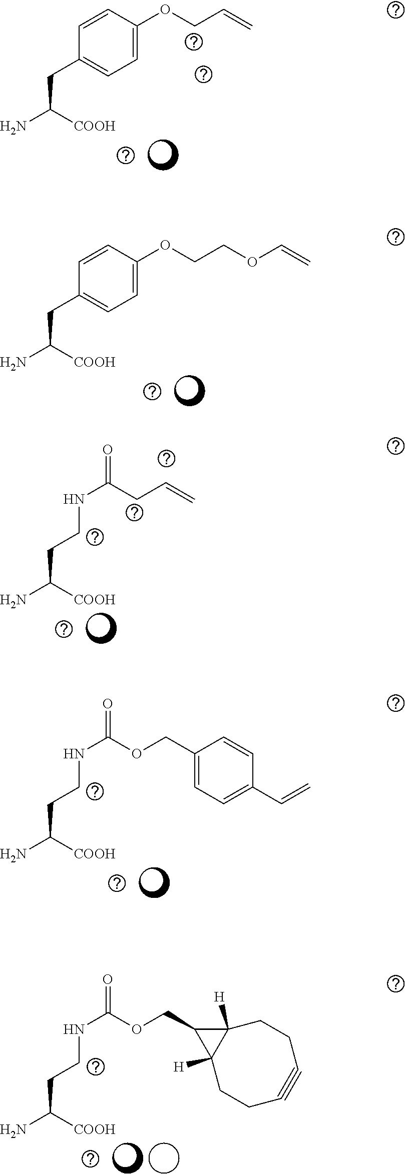 [SMILES: C=CCOc1ccc(C[C@H](N)C(=O)O)cc1 ; C=COCCOc1ccc(C[C@H](N)C(=O)O)cc1 ; C=CCC(=O)NCC[C@H](N)C(=O)O ; C=Cc1ccc(COC(=O)NCC[C@H](N)C(=O)O)cc1 ; N[C@@H](CCNC(=O)OC[C@H]1[C@@H]2CCC#CCC[C@@H]21)C(=O)O]

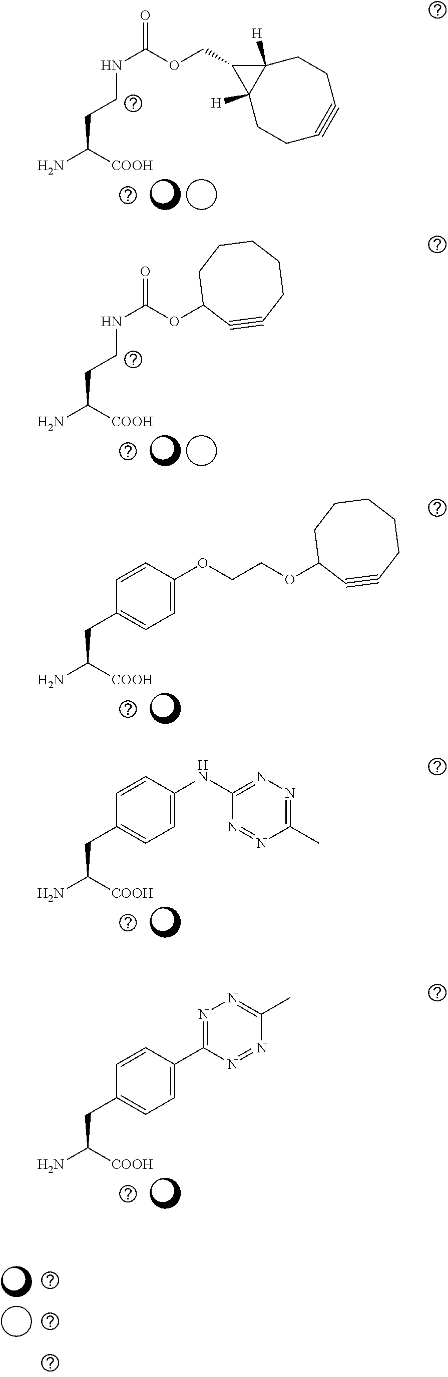 [SMILES: N[C@@H](CCNC(=O)OC[C@@H]1[C@@H]2CCC#CCC[C@@H]21)C(=O)O ; N[C@@H](CCNC(=O)OC1C#CCCCCC1)C(=O)O ; N[C@@H](Cc1ccc(OCCOC2C#CCCCCC2)cc1)C(=O)O ; Cc1nnc(Nc2ccc(C[C@H](N)C(=O)O)cc2)nn1 ; Cc1nnc(-c2ccc(C[C@H](N)C(=O)O)cc2)nn1]

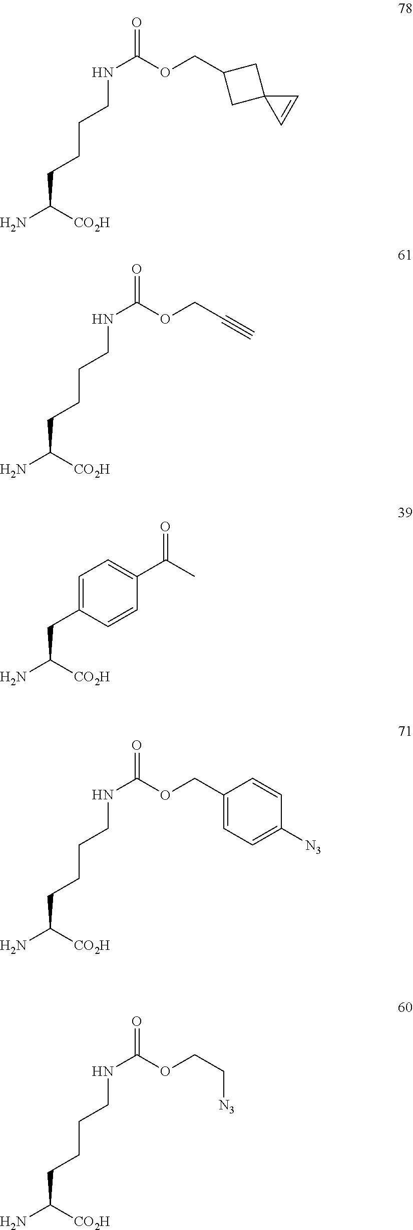 [SMILES: N[C@@H](CCCCNC(=O)OCC1CC2(C=C2)C1)C(=O)O ; C#CCOC(=O)NCCCC[C@H](N)C(=O)O ; CC(=O)c1ccc(C[C@H](N)C(=O)O)cc1 ; [N-]=[N+]=Nc1ccc(COC(=O)NCCCC[C@H](N)C(=O)O)cc1 ; [N-]=[N+]=NCCOC(=O)NCCCC[C@H](N)C(=O)O]

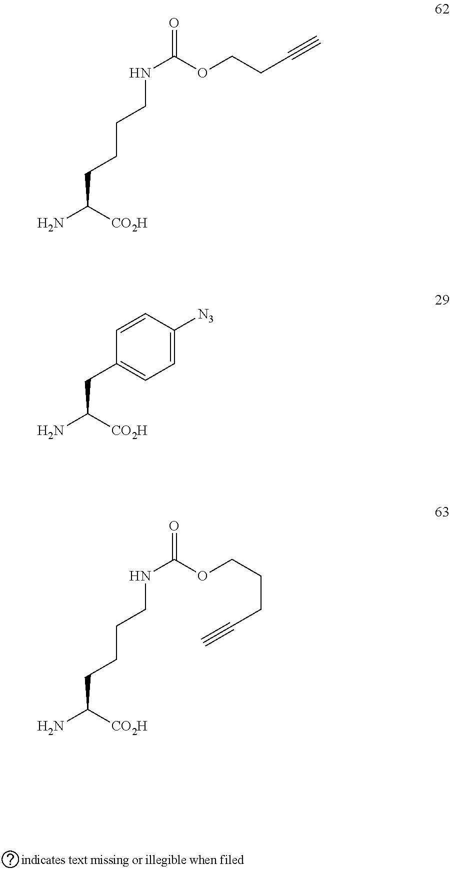 [SMILES: C#CCCOC(=O)NCCCC[C@H](N)C(=O)O ; [N-]=[N+]=Nc1ccc(C[C@H](N)C(=O)O)cc1 ; C#CCCCOC(=O)NCCCC[C@H](N)C(=O)O]

19. The method of claim 15, wherein the unnatural amino acid that includes the conjugation handle has a structure of

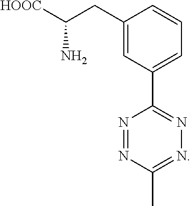 [SMILES: Cc1nnc(-c2cccc(C[C@H](N)C(=O)O)c2)nn1]

20. The method of claim 15, wherein bioconjugating the compound with the protein of interest at least partially degrades the protein of interest.

21. The method of claim 15, wherein bioconjugating the compound with the protein of interest promotes degradation of the protein of interest via a ubiquitin proteosome system.
